(19) United States
(12) Patent Application Publication
(10) Pub. No.: US 2010/0311800 A1
(43) Pub. Date: Dec. 9, 2010

(54) ANGIOTENSIN II RECEPTOR ANTAGONISTS

(76) Inventors: Nicoletta Almirante, Milano (IT); Alessia Nicotra, Grandate (Como) (IT); Silvia Stefanini, San Donato Milanese (Milano) (IT); Stefano Biondi, Pero (Milano) (IT); Ennio Ongini, Segrate (Milano) (IT); Amjad Ali, Freehold, NJ (US); Michael Man-chu Lo, Edison, NJ (US)

Correspondence Address: ARENT FOX LLP, 1050 CONNECTICUT AVENUE, N.W., SUITE 400, WASHINGTON, DC, 20036, US

(21) Appl. No.: 12/867,050

(22) PCT Filed: Feb. 19, 2009
(86) PCT No.: PCT/EP09/51942

§ 371 Date: Aug. 10, 2010

Related U.S. Application Data
(60) Provisional application No. 61/193,347, filed on Nov. 20, 2008.
(60) Provisional application No. 61/064,275, filed on Feb. 26, 2008.

(51) Int. Cl. A61K 31/41; C07D 257/04; A61P 9/12

(52) U.S. Cl. 514/381; 548/253

(57) ABSTRACT

The present invention relates to nitrooxyderivatives of angiotensin II receptor antagonists.

The present invention refers to angiotensin II receptor antagonist nitrooxyderivatives and salts thereof, to the use of these compounds for treating hypertension and related diseases and to pharmaceutical formulations for controlled and sustained delivery of these compounds to a patient.

U.S. Pat. No. 5,138,069 generically and specifically describes 2-butyl-4-chloro-1-[p-(o-1H-tetrazol-5-ylphenyl)-benzyl]imidazole-5-methanol potassium salt and 2-butyl-4-chloro-1-[(2′-1H-tetrazol-5-yl)biphenyl-4-yl)methyl]imidazole-5-carboxylic acid. Columns 261-263 of U.S. Pat. No. 5,136,069 describe general procedures for formulating compounds described in the patent, including capsules, tablets, injection formulations, and suspensions. U.S. Pat. No. 5,153,197, describes the use of these compounds, alone and in combination with a diuretic, to treat a patient having hypertension.

WO2005011646 describes angiotensin II receptor blocker nitroderivatives, pharmaceutical compositions containing them and their use for the treatment of cardiovascular, renal and chronic liver diseases, inflammatory processes and metabolic syndromes. The publication describes a variety of angiotensin receptor blocker compounds each of which are covalently linked in a variety of ways to a nitric oxide group. Specific examples include angiotensin receptor blockers with one covalently-linked nitric oxide group, and angiotensin receptor blockers with two independently-covalently-linked nitric oxide groups.

WO2005023182 describes nitrosated and nitrosylated cardiovascular compounds, and compositions comprising at least one nitrosated and nitrosylated cardiovascular compound and optionally at least one nitric oxide donor. The cardiovascular compound, which is nitrosated or nitrosylated, may be an aldosterone antagonist, an angiotensin II receptor antagonist, a calcium channel blocker, an endothelin antagonist, a hydralazine compound, a neutral endopeptidase inhibitor or a renin inhibitor. The nitric oxide donor may be selected from S-nitrosothiols, nitrites, nitrates, N-oxo-N-nitrosamines, furoxans, and sydnonimines.

WO2005070868 describes combination therapy for treating cyclooxygenase-2 mediated diseases or conditions at risk of thrombotic cardiovascular events, which involves administering selected cyclooxygenase-2 inhibitor in combination with a nitric oxide donating compound such as 5,6-bis(nitrooxy)hexyl acetate, 6-hydroxyhexane-1,2-diyl dinitrate, 5-hydroxypentane-1,2-diyl dinitrate, (5R)-5,6-bis(nitrooxy)hexyl 4-nitrobenzoate, (5S)-5,6-bis(nitrooxy)hexyl 4-nitrobenzoate, (2R)-6-hydroxyhexane-1,2-diyl dinitrate, (2S)-6-hydroxyhexane-1,2-diyl dinitrate, (2S)-propane-1,2-diyl dinitrate, and (2R)-propane-1,2-diyl dinitrate.

The present invention relates to angiotensin II receptor antagonist nitrooxyderivatives, including 2-butyl-4-chloro-1-[(2′-(1-H-tetrazol-5-yl)biphenyl-4-yl)methyl]-imidazole-5-carboxylate nitrooxyderivatives, including various pharmaceutically acceptable salts and hydrates of these forms, and pharmaceutical formulations for controlled and sustained delivery of these forms to a patient.

The salts include non-toxic salts such as those derived from inorganic acids, e.g. hydrochloric, hydrobromoic, sulfuric, sulfamic, phosphoric, nitric and the like, or the quaternary ammonium salts which are formed, e.g., from inorganic or organic acids or bases. Examples of acid addition salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, nitrate, oxalate, pamoate, pectinate, persulfate, 3-phenylpropionate, picrate, pivalate, propionate, succinate, sulfate, tartrate, thiocyanate, tosylate, and undecanoate. Base salts include ammonium salts, alkali metal salts such as sodium and potassium salts, alkaline earth metal salts such as calcium and magnesium salts, salts with organic bases such as dicyclohexylamine salts, N-methyl-D-glucamine, and salts with amino acids such as arginine, lysine, and so forth. Also, the basic nitrogen-containing groups may be quaternized with such agents as lower alkyl halides, such as methyl, ethyl, propyl, and butyl chloride, bromides and iodides; dialkyl sulfates like dimethyl, diethyl, dibutyl; and diamyl sulfates, long chain halides such as decyl, lauryl, myristyl and stearyl chlorides, bromides and iodides, aralkyl halides like benzyl and phenethyl bromides and others.

The invention also includes a method for treating hypertension, congestive heart failure, pulmonary hypertension, renal insufficiency, renal ischemia, renal failure, renal fibrosis, cardiac insufficiency, cardiac hypertrophy, cardiac fibrosis, myocardial ischemia, cardiomyopathy, glomerulonephritis, renal colic, complications resulting from diabetes such as nephropathy, vasculopathy and neuropathy, glaucoma, elevated intra-ocular pressure, atherosclerosis, restenosis post angioplasty, complications following vascular or cardiac surgery, erectile dysfunction, hyperaldosteronism, lung fibrosis, scleroderma, anxiety, cognitive disorders, complications of treatments with immunosuppressive agents, and other diseases known to be related to the renin-angiotensin system, by administering an angiotensin II receptor antagonist of the invention to a patient having one or more of these conditions.

### DETAILED DESCRIPTION OF THE INVENTION AND PREFERRED EMBODIMENTS

Compounds of the invention are angiotensin II receptor antagonist nitrooxyderivatives having the general formula (I):

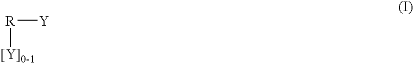

wherein R is selected from the group consisting of (IIa)-(IIk):

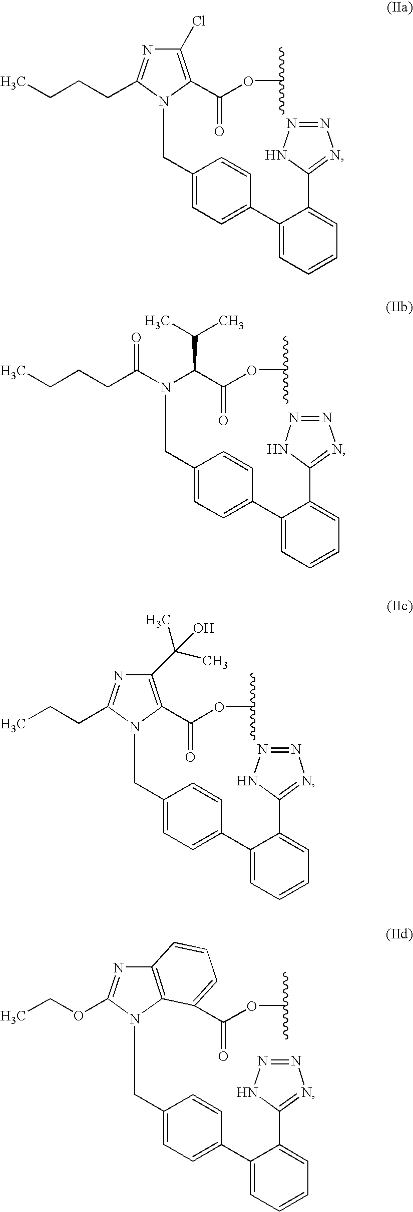

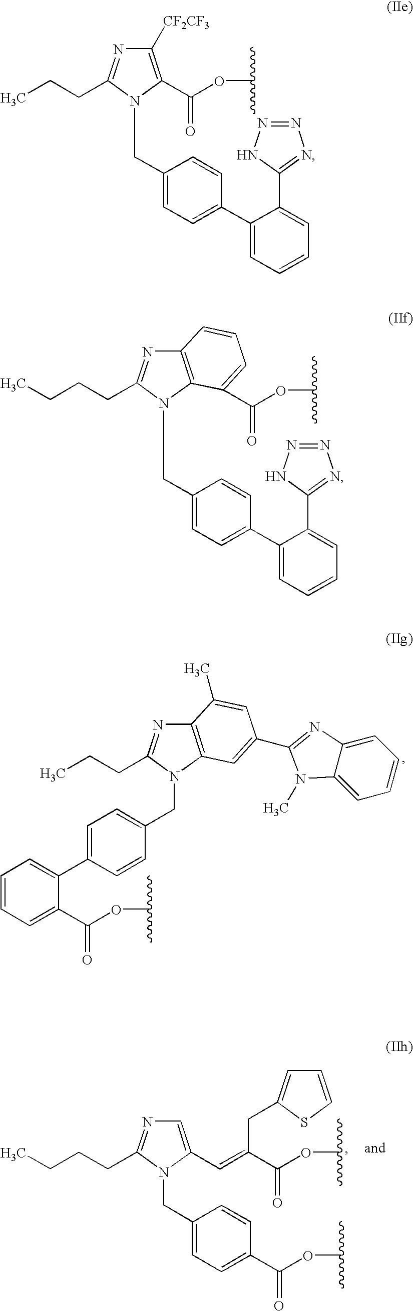

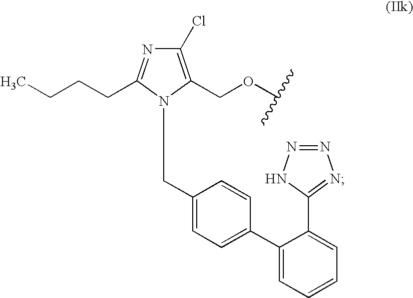

when R is selected from (IIk), Y is selected from the group consisting of:

1) —C(O)(CH₂)ₙR⁵

2) —C(O)(CH₂)ₙ—O—CH₂—R⁵,

3) —C(O)—R⁶ wherein R⁶ is the following group:

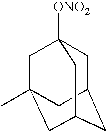

when R is selected from (IIa)-(IIh), Y is selected from the group consisting of:

4) —C(R¹R²)OC(O)—(CH₂)ₙR⁵,

5) —C(R¹R²)OC(O)O—(CH₂)ₙR⁵,

6) C(R¹R²)OC(O)(CH₂)ₙ—O—CH₂—R⁵,

7) C(R¹R²)OC(O)O(CH₂)ₙ—O—CH₂—R⁵

8) —C(R¹R²)OC(O)—R⁶ wherein R⁶ is as above defined;

R¹ and R² are independently selected from the group consisting of hydrogen and C₁₋₄ alkyl;

### R⁵ is —CH(ONO₂)R⁷;

R⁷ is CH₃ or C₁₋₄ alkyl;
n is an integer from 1 to 4;
or a pharmaceutically acceptable salt thereof.

In one embodiment, R¹ is CH₃ and R² is H or CH₃ and all other variables are as previously defined.

In another embodiment, R is selected from the group consisting of (IIb), (IIc) and (IIg):

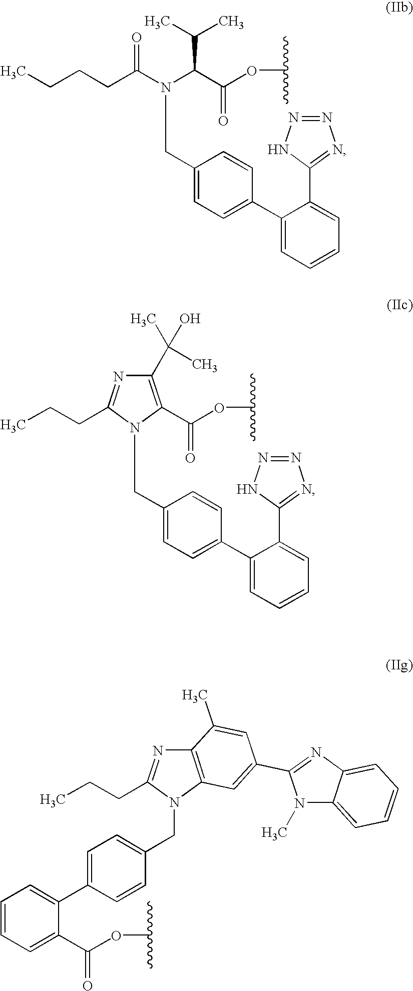

In another embodiment, R is selected from the group consisting of (IIa), (IId), and (IIk):

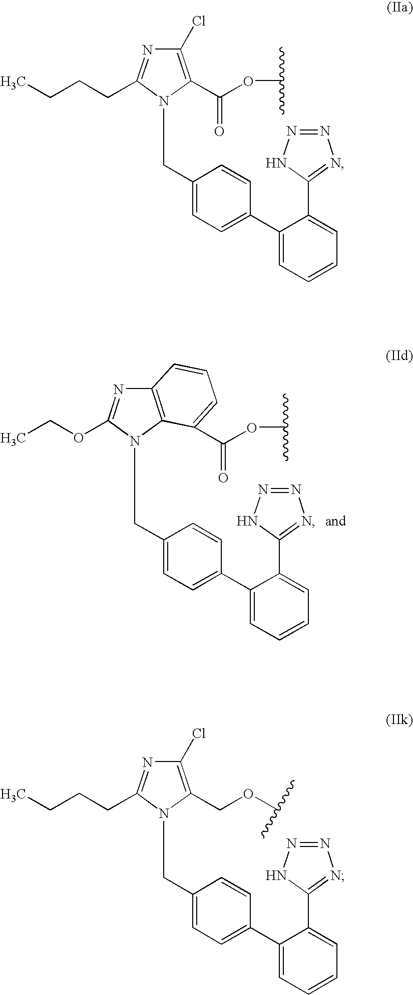

and all other variables are as previously defined.

In another embodiment, R⁵ is selected from the group consisting of

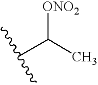

and all other variables are as previously defined.

In another embodiment, R⁵ is selected from the group consisting of

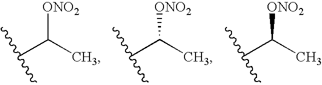

and all other variables are as previously defined.

In another embodiment, the compound is selected from the group of compounds (1) to (18) shown below:

TABLE (1)-(9)

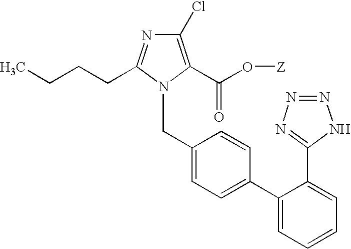

Z

| | |
|---|---|
| 1) | 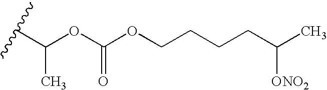 |
| 2) | 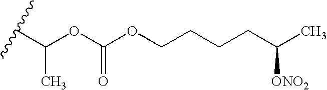 |
| 3) | 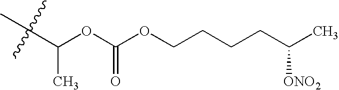 |
| 4) | 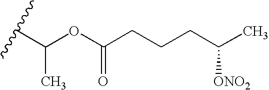 |
| 5) | 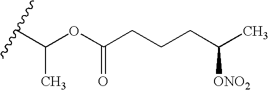 |
| 6) | 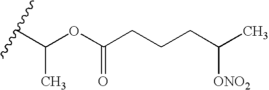 |
| 7) | 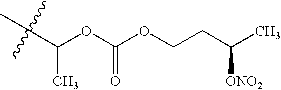 |
| 8) | 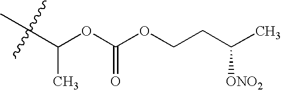 |
| 9) | 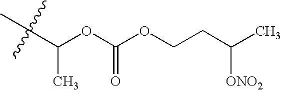 |

TABLE (10)-(18)

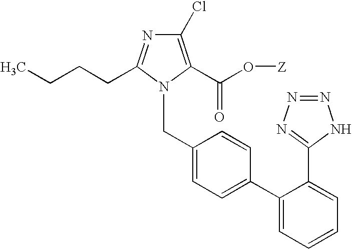

Z

| | |
|---|---|
| 10) | 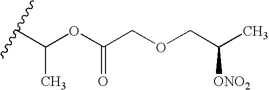 |
| 11) | 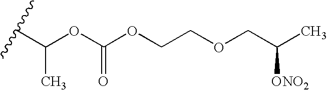 |
| 12) | 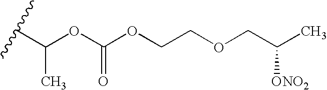 |
| 13) | 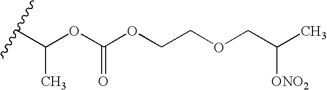 |
| 14) | 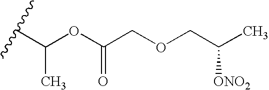 |
| 15) | 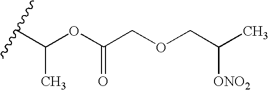 |
| 16) | 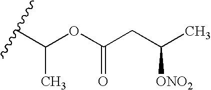 |
| 17) | 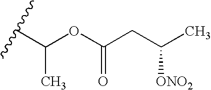 |
| 18) | 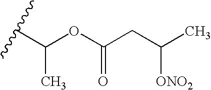 |

In another embodiment, the compound is selected from the group of compounds (19) to (36) shown below:

TABLE (19)-(27)

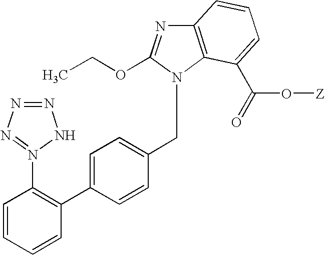

Z

| | |
|---|---|
| 19) | 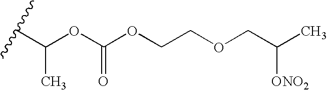 |
| 20) | 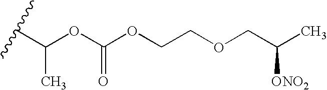 |
| 21) | 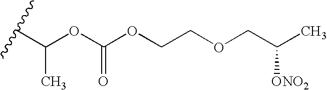 |
| 22) | 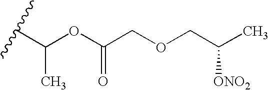 |
| 23) | 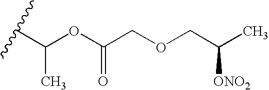 |
| 24) | 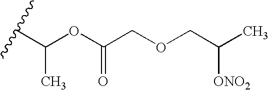 |
| 25) | 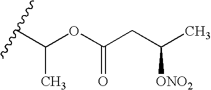 |
| 26) | 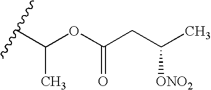 |
| 27) | 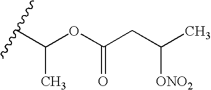 |

TABLE (28)-(36)

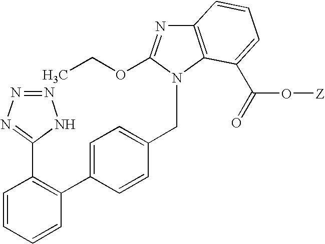

Z

| | |
|---|---|
| 28) | 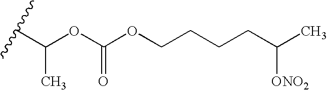 |
| 29) | 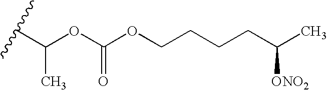 |
| 30) | 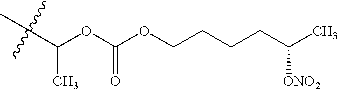 |
| 31) | 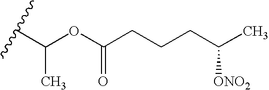 |
| 32) | 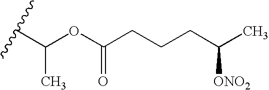 |
| 33) | 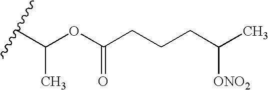 |
| 34) | 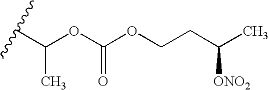 |
| 35) | 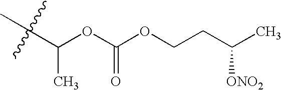 |
| 36) | 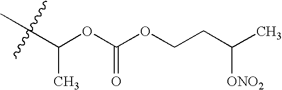 |

In another embodiment, the compound is selected from the group of compounds (37) to (45) shown below:

TABLE (37)-(45)

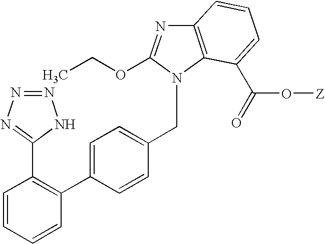

Z

| | |
|---|---|
| 37) | 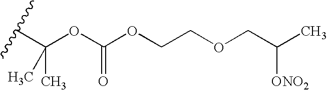 |
| 38) | 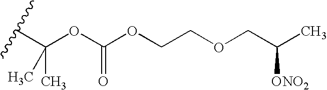 |
| 39) | 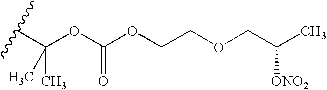 |
| 40) | 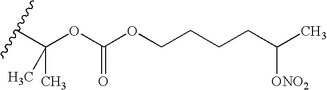 |
| 41) | 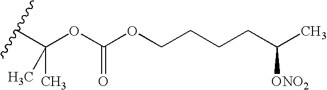 |
| 42) | 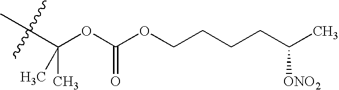 |
| 43) | 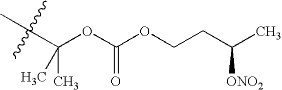 |
| 44) | 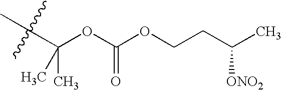 |
| 45) | 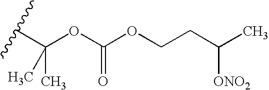 |

In another embodiment, the compound is selected from the group of compounds (46) to (52) shown below:

TABLE (46)-(52)

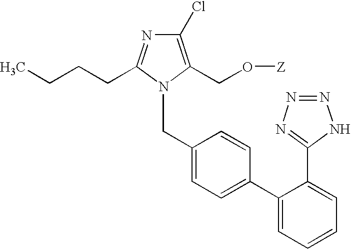

Z

| | |
|---|---|
| 46) | 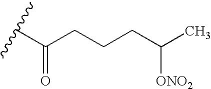 |
| 47) | 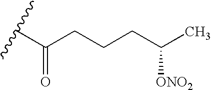 |
| 48) | 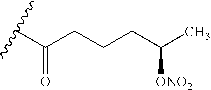 |
| 49) | 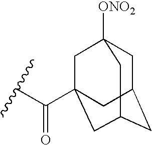 |
| 50) | 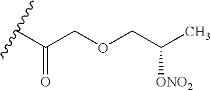 |
| 51) | 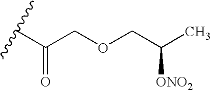 |
| 52) | 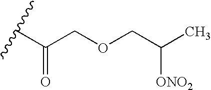 |

The compounds of the present invention may have one or more chiral centers, providing more stereoisomers. This invention includes all of the stereoisomers and mixtures thereof. Unless specifically mentioned otherwise, reference to one stereoisomer applies to any of the possible stereoisomers. Whenever the stereoisomeric composition is unspecified, all possible stereoisomers are included. The structure marking “*” indicates the location of a carbon atom that is a chiral center.

As used herein except where noted, “alkyl” is intended to include both branched- and straight-chain saturated aliphatic hydrocarbon groups having the specified number of carbon atoms. Commonly used abbreviations for alkyl groups are used throughout the specification, e.g. methyl may be represented by conventional abbreviations including “Me” or CH₃ or a symbol that is an extended bond as the terminal group, e.g.

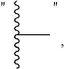

ethyl may be represented by “Et” or CH₂CH₃, propyl may be represented by “Pr” or CH₂CH₂CH₃, butyl may be represented by “Bu” or CH₂CH₂CH₂CH₃, etc. “C₁₋₄ alkyl” (or “C₁-C₄ alkyl”) for example, means linear or branched chain alkyl groups, including all isomers, having the specified number of carbon atoms. C₁₋₄ alkyl includes n-, iso-, sec- and t-butyl, n- and isopropyl, ethyl and methyl. If no number is specified, 1-4 carbon atoms are intended for linear or branched alkyl groups.

### Assay on Vascular Tone

The ability of the compounds of the invention to induce vasorelaxation in comparison to native angiotensin II receptor blockers (ARBs) was tested in vitro in isolated rabbit thoracic aorta preparations (Wanstall J. C. et al., Br. J. Pharmacol., 134:463-472, 2001). Male New Zealand rabbits were anaesthetized with thiopental-Na (50 mg/kg, iv), sacrificed by exsanguinations and then the thorax was opened and the aorta dissected. Aortic ring preparations (4 mm in length) were set up in physiological salt solution (PSS) at 37° C. in small organ chambers (5 ml). The composition of PSS was (mM): NaCl 130, NaHCO₃ 14.9, KH₂PO₄ 1.2, MgSO₄ 1.2, HEPES 10, CaCl₂, ascorbic acid 170 and glucose 1.1 (95% O₂/5% CO₂; pH 7.4). Each ring was mounted under 2 g passive tension. Isometric tension was recorded with a Grass transducer (Grass FT03) attached to a BIOPAC MP150 System. Preparations were allowed to equilibrate for 1 h, and then contracted submaximally with noradrenaline (NA, 1 μM) and, when the contraction was stable, acetylcholine (ACh, 10 μM) was added. A relaxant response to ACh indicated the presence of a functional endothelium. Vessels that were unable to contract NA or showed no relaxation to Ach were discarded. When a stable precontraction was reached, a cumulative concentration-response curve to either of the vasorelaxant agents was obtained in the presence of a functional endothelium. Each arterial ring was exposed to only one combination of inhibitor and vasorelaxant. Moreover, the effect of the soluble guanylyl cyclase inhibitor ODQ (1-H-(1,2,4)-oxadiazol(4,3-a)quinoxalin-1-one) on vasorelaxation elicited by the compounds was examined preincubating the aortic rings with ODQ (10 μM) for 20 min.

Responses to relaxing agents are expressed as a percentage of residual contraction and plotted against concentration of test compound. EC₅₀ values (where EC₅₀ is the concentration producing 50% of the maximum relaxation to the test compound) were interpolated from these plots. During the experimental period, the plateau obtained with NA was stable without significant spontaneous loss of contraction in the aortic rings. Under these experimental conditions, the native ARBs did not produce relaxation at any of the concentration tested, the curve being not different from that built up in the presence of vehicle alone.

As shown in Table 1, the compounds of the invention were able to induce relaxation in a concentration-dependent manner. Furthermore, in experiments performed in the presence of ODQ (10 μM), the vasorelaxant responses to tested compounds were inhibited.

Data Table 1

| | Compound | EC₅₀ in vessel |
|---|---|---|
| Structure | Number | relaxation assay |
| 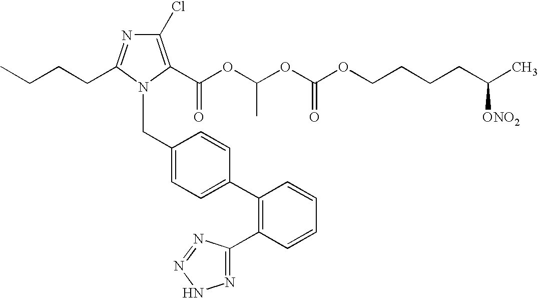 | Example 1 | 22 μM |
| 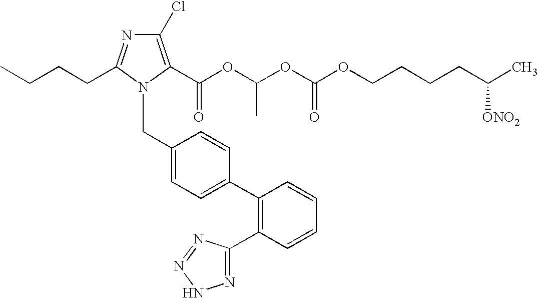 | Example 3 | 39 μM |
| 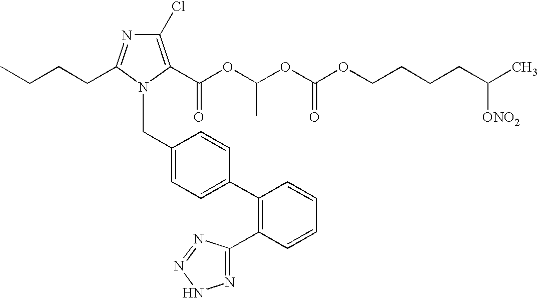 | Example 4 | 27 μM |
| 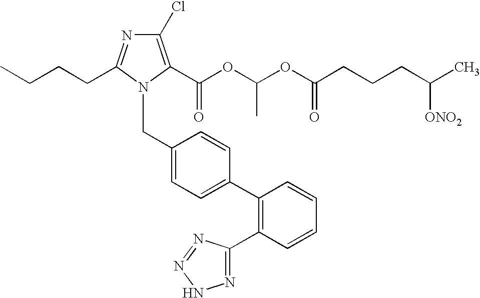 | Example 8 | 40 μM |
| 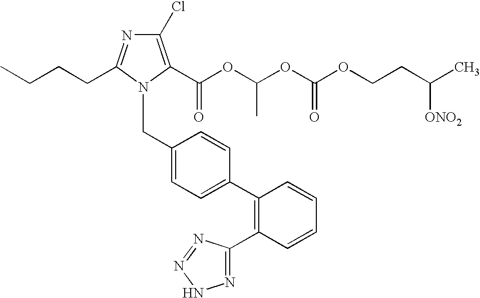 | Example 5 | 22 μM |
| 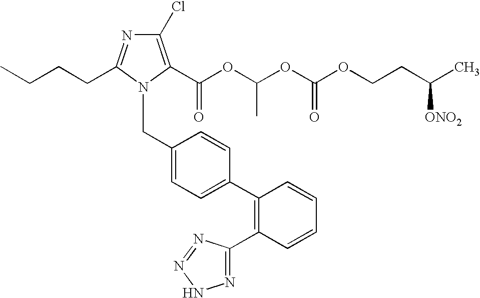 | Example 6 | 23 μM |
| 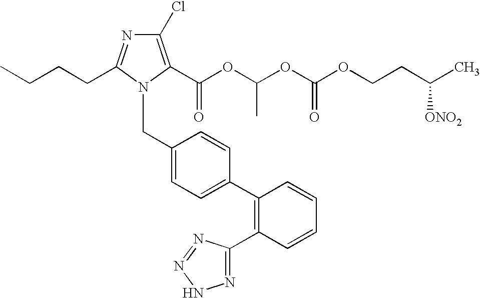 | Example 7 | 26 μM |
| 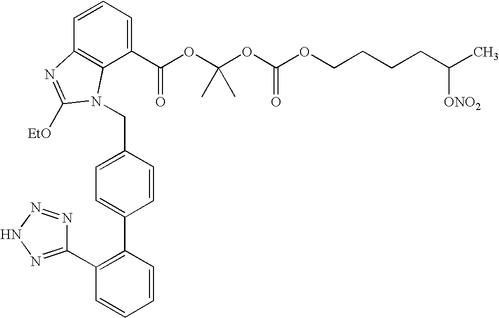 | Example 2 | 31 μM |
| 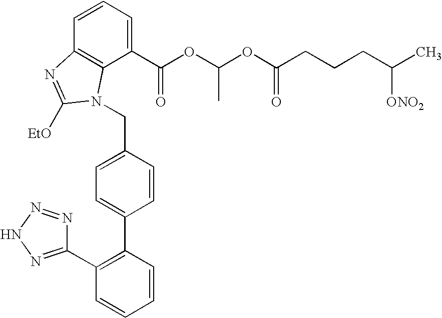 | Example 9 | 43 μM |
| 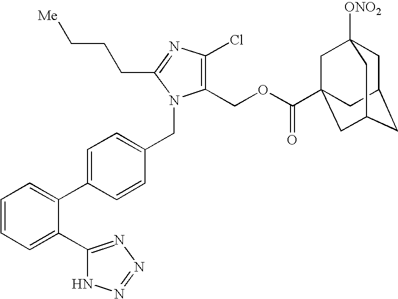 | Example 10 | 5.3 μM |

### Assay for Reactive Nitrogen Species (RNS) (DAN Assay)

RNS were detected as S-nitrosothiols (RNSOs) in EDTA-treated rat plasma using an HPLC fluorescent assay based on the method of Kostka and Park (Methods Enzymol. 1999, 301, 227-235). The method is based on the detection of fluorescent 2,3-naphthotriazole (NAT) formed in the reaction between acidified 2,3-diaminonaphthalene (DAN) and the nitrosonium moiety of RSNOs released by HgCl₂-mediated breakdown of the S—NO bond. The reaction mixture was chromatographed by reversed phase HPLC, and the fluorescent signal of the resolved NAT peak was quantified.

Plasma (20 μL) was first diluted 1:1 in H₂O (20 μL) in a black polypropylene untreated microtiter plate. DAN reagent (100 μL per well, 100 μM DAN in 0.1 N HCl, 4 mM HgCl₂) was added, and the plate was immediately sealed with an opaque plate mat, vortexed, and incubated in the dark for 10 min. Plates were centrifuged (2000×g, 5 min) and chilled to 4° C. before HPLC analysis. HPLC was carried out on an Agilent 1200 system using a chilled autosampler (4° C.). Samples were chromatographed on a C8 column (Zorbax Eclipse XDB-C8, 4.6×150 mm, 5 μm) with isocratic elution using a mobile phase of 67% MeOH, 0.1% NH₄OAc and a flow rate of 2 mL/min. NAT fluorescence was monitored at 450 nm using an excitation wavelength of 360 nm. Calibration curves were prepared using NaNO₂ in control plasma. Typical range of quantitation was 0.1 μM to 30 μM NO₂.

Compared to Compound A, the compound of the invention (Example 4) showed improved RNS levels (see Data Table 2).

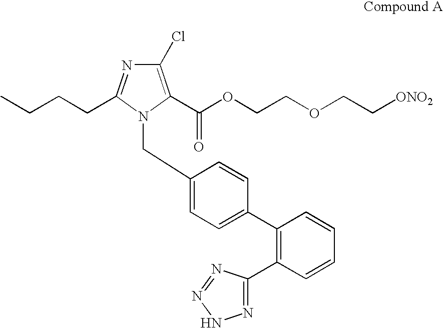

DATA TABLE 2
RNS levels - Compound A and Example 4

| Time(h) | Compound A | Example 4 |
|---|---|---|
| 0 | 0.4 | 0.9 |
| 1 | 2.8 | 6.9 |
| 3 | 1.4 | 6.9 |
| 6 | 0.9 | 2.4 |
| 24 | 0 | 1.6 |

### Assay for Antihypertensive Activity (In Vivo)

The ability of the compounds of the invention to decrease blood pressure was evaluated in conscious spontaneously hypertensive rats (SHRs). SHRs (250-300 g) received a single oral dose of tested compounds. Systolic blood pressure (SBP) and heart rate were monitored by telemetry for 24 hours after dosing. SBP was evaluated before (baseline) and at different time points (i.e. 2-6, 12, 21-hours) following treatment by oral administration of the compounds. The data were processed both as the absolute value or as a delta between the absolute value and its own baseline.

The Dataquest IV telemetry system (Data Sciences International) was used for measurement of systolic pressure, diastolic pressure, mean arterial pressure, heart rate, and motor activity. The monitoring system consists of a transmitter (radio frequency transducer model TA11PA), receiver panel, consolidation matrix, and personal computer with accompanying software. Before the device was implanted, calibrations were verified to be accurate within ±3 mmHg. Rats were anesthetized with ketamine/xylazine/acepromazine, and the flexible catheter of the transmitter was surgically secured in the abdominal aorta just below the renal arteries. The transmitter was sutured subcutaneously. Rats were housed in individual cages after the operation. Each cage was placed over the receiver panel that was connected to the personal computer for data acquisition. The rats were unrestrained and free to move within their cages. Hemodynamic data were sampled every 2 minutes for 10 seconds.

Compared to Compound A, the compounds of the invention (Examples 2, 4, 6, 8 and 9) provided BP lowering with extended peak effect and duration of action at the same dose (10 mg/kg) or at an inferior dose (3 mg/kg) (see Data Table 3).

DATA TABLE 3
Δ SBP - Compound A and Examples 2, 4, 6, 8 and 9

| | Δ SBP in SHR (mmHg) | | |
|---|---|---|---|
| Compound | 2-6 h | 12 h | 21-24 h |
| A | −15 | −12 | −8 |
| (10 mpk) | | | |
| Example 2 | −30 | −34 | −36 |
| (3 mpk) | | | |
| Example 4 | −10 | −30 | −30 |
| (10 mpk) | | | |
| Example 6 | −16 | −21 | −17 |
| (10 mpk) | | | |
| Example 8 | −31 | −29 | −28 |
| (10 mpk) | | | |
| Example 9 | −43 | −49 | −50 |
| (3 mpk) | | | |

The angiotensin II receptor antagonists (ARBs) of the invention are useful for the treatment and/or prophylaxis of diseases which are related to hypertension, congestive heart failure, pulmonary hypertension, renal insufficiency, renal ischemia, renal failure, renal fibrosis, cardiac insufficiency, cardiac hypertrophy, cardiac fibrosis, myocardial ischemia, cardiomyopathy, glomerulonephritis, renal colic, complications resulting from diabetes such as nephropathy, vasculopathy and neuropathy, glaucoma, elevated intra-ocular pressure, atherosclerosis, restenosis post angioplasty, complications following vascular or cardiac surgery, erectile dysfunction, hyperaldosteronism, lung fibrosis, scleroderma, anxiety, cognitive disorders, complications of treatments with immunosuppressive agents, and other diseases known to be related to the renin-angiotensin system.

The ARBs of the invention are especially useful for the treatment and/or prophylaxis of diseases which are related to hypertension, congestive heart failure, pulmonary hypertension, renal insufficiency, renal ischemia, renal failure, renal fibrosis, cardiac insufficiency, cardiac hypertrophy, cardiac fibrosis, myocardial ischemia, cardiomyopathy, complications resulting from diabetes such as nephropathy, vasculopathy and neuropathy.

In one embodiment, the invention relates to a method for the treatment and/or prophylaxis of diseases, which are associated with a dysregulation of the renin-angiotensin system, in particular to a method for the treatment or prophylaxis of the above-mentioned diseases, said methods comprising administering to a patient a pharmaceutically active amount of an angiotensin II receptor antagonist of the invention.

The invention also relates to the use of ARBs of the invention for the preparation of a medicament for the treatment and/or prophylaxis of the above-mentioned diseases.

The above-mentioned ARBs of the invention are also of use in combination with other pharmacologically active compounds comprising angiotensin converting enzyme inhibitors (e.g, alacepril, benazepril, captopril, ceronapril, cilazapril, delapril, enalapril, enalaprilat, fosinopril, imidapril, lisinopril, moveltipril, perindopril, quinapril, ramipril, spirapril, temocapril, or trandolapril), neutral endopeptidase inhibitors (e.g., thiorphan and phosphoramidon), aldosterone antagonists, renin inhibitors (e.g. urea derivatives of di- and tri-peptides (See U.S. Pat. No. 5,116,835), amino acids and derivatives (U.S. Pat. Nos. 5,095,119 and 5,104,869), amino acid chains linked by non-peptidic bonds (U.S. Pat. No. 5,114,937), di- and tri-peptide derivatives (U.S. Pat. No. 5,106,835), peptidyl amino diols (U.S. Pat. Nos. 5,063,208 and 4,845,079) and peptidyl beta-aminoacyl aminodiol carbamates (U.S. Pat. No. 5,089,471); also, a variety of other peptide analogs as disclosed in the following U.S. Pat. Nos. 5,071,837; 5,064,965; 5,063,207; 5,036,054; 5,036,053; 5,034,512 and 4,894,437, and small molecule renin inhibitors (including diol sulfonamides and sulfinyls (U.S. Pat. No. 5,098,924), N-morpholino derivatives (U.S. Pat. No. 5,055,466), N-heterocyclic alcohols (U.S. Pat. No. 4,885,292) and pyrolimidazolones (U.S. Pat. No. 5,075,451); also, pepstatin derivatives (U.S. Pat. No. 4,980,283) and fluoro- and chloro-derivatives of statone-containing peptides (U.S. Pat. No. 5,066,643), enalkrein, RO 42-5892, A 65317, CP 80794, ES 1005, ES 8891, SQ 34017, aliskiren ((2S,4S,5S,7S)—N-(2-carbamoyl-2-methylpropyl)-5-amino-4-hydroxy-2,7-diisopropyl-8-[4-methoxy-3-(3-methoxypropoxy)phenyl]-octanamid hemifumarate) SPP600, SPP630 and SPP635), endothelin receptors antagonists, vasodilators, calcium channel blockers (e.g., amlodipine, nifedipine, veraparmil, diltiazem, gallopamil, niludipine, nimodipins, nicardipine), potassium channel activators (e.g., nicorandil, pinacidil, cromakalim, minoxidil, aprilkalim, loprazolam), diuretics (e.g., hydrochlorothiazide), sympatholitics, beta-adrenergic blocking drugs (e.g., propranolol, atenolol, bisoprolol, carvedilol, metoprolol, or metoprolol tartate), alpha adrenergic blocking drugs (e.g., doxazocin, prazosin or alpha methyldopa) central alpha adrenergic agonists, peripheral vasodilators (e.g. hydralazine), lipid lowering agents (e.g., simvastatin, lovastatin, ezetimibe, atorvastatin, pravastatin), metabolic altering agents including insulin sensitizing agents and related compounds (e.g., muraglitazar, glipizide, metformin, rosiglitazone)) or with other drugs beneficial for the prevention or the treatment of the above-mentioned diseases including nitroprusside and diazoxide.

The dosage regimen utilizing the angiotensin II receptor antagonists is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter, or arrest the progress of the condition.

Oral dosages of the angiotensin II receptor antagonists, when used for the indicated effects, will range between about 0.0125 mg per kg of body weight per day (mg/kg/day) to about 7.5 mg/kg/day, preferably 0.0125 mg/kg/day to 3.75 mg/kg/day, and more preferably 0.3125 mg/kg/day to 1.875 mg/kg/day. For example, an 80 kg patient would receive between about 1 mg/day and 600 mg/day, preferably 1 mg/day to 300 mg/day, and more preferably 25 mg/day to 150 mg/day. A suitably prepared medicament for once a day administration would thus contain between 1 mg and 600 mg, preferably between 1 mg and 300 mg, and more preferably between 25 mg and 300 mg, e.g., 25 mg, 50 mg, 100 mg, 150, 200, 250 and 300 mg. Advantageously, the angiotensin II receptor antagonists may be administered in divided doses of two, three, or four times daily. For administration twice a day, a suitably prepared medicament would contain between 0.5 mg and 300 mg, preferably between 0.5 mg and 150 mg, more preferably between 12.5 mg and 150 mg, e.g., 12.5 mg, 25 mg, 50 mg, 75 mg, 100 mg, 125 mg and 150 mg.

The angiotensin II receptor antagonists of the invention can be administered in such oral forms as tablets, capsules and granules. The angiotensin II receptor antagonists are typically administered as active ingredients in admixture with suitable pharmaceutical binders as described below. % w/w expresses the weight percent of the indicated composition constituent compared to the total composition. Suitable fillers used in these dosage forms include microcrystalline cellulose, silicified microcrystalline cellulose, dicalcium phosphate, lactose, mannitol, and starch, preferably microcrystalline cellulose, dicalcium phosphate, lactose or mixtures thereof. Suitable binders include hydroxypropyl cellulose, hydroxypropyl methyl cellulose, starch, gelatin, natural sugars such as glucose or beta-lactose, corn-sweeteners, natural and synthetic gums such as acacia, tragacanth or sodium alginate, carboxymethylcellulose, and polyvinyl pyrrolidone. Lubricants used in these dosage forms include sodium oleate, sodium stearate, magnesium stearate, sodium benzoate, sodium acetate, sodium chloride, sodium stearyl fumarate, stearic acid and the like, preferably magnesium stearate. Suitable coating compositions include aqueous dispersion or organic solution of insoluble polymers such as ethyl cellulose, cellulose aetate, cellulose acetate butyrate and acrylate copolymers commercially known as Eudragit®. Plasticizers include triethyl citrate, dibutyl sebacate, dibutyl phthalate, triacetin and castor oil. Antitacking agents include talc, kaolin, colloidal silica or mixtures thereof.

2-Butyl-4-chloro-1-[(2′-(1-H-tetrazol-5-yl)biphenyl-4-yl)methyl]-imidazole-5-carboxylic acid is the active metabolite of 2-butyl-4-chloro-1-[p-(o-1H-tetrazol-5-ylphenyl)-benzyl]imidazole-5-methanol which is available as a monopotassium salt (also known as losartan potassium salt). Losartan potassium salt is available commercially as the active ingredient in COZAAR® (Merck & Co., Inc. Whitehouse Station, N.J.). The preparation of losartan potassium salt is described in U.S. Pat. Nos. 5,138,069, 5,130,439, and 5,310,928. Tetrazolylphenylboronic acid intermediates useful in the synthesis of losartan potassium salt are described in U.S. Pat. No. 5,206,374. Additional patents that describe procedures useful for making losartan include U.S. Pat. Nos. 4,820,843, 4,870,186, 4,874,867, 5,039,814, and 5,859,258.

### General Synthesis

The compound of general formula (I)

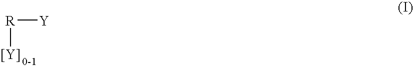

wherein R and Y are as above defined, can be prepared as follow:
1.a) R is selected from the group consisting of (IIa)-(IIh); R² is H; R¹≠R²
by a process comprising reacting a compound of formula (IIIa)-(IIIh)

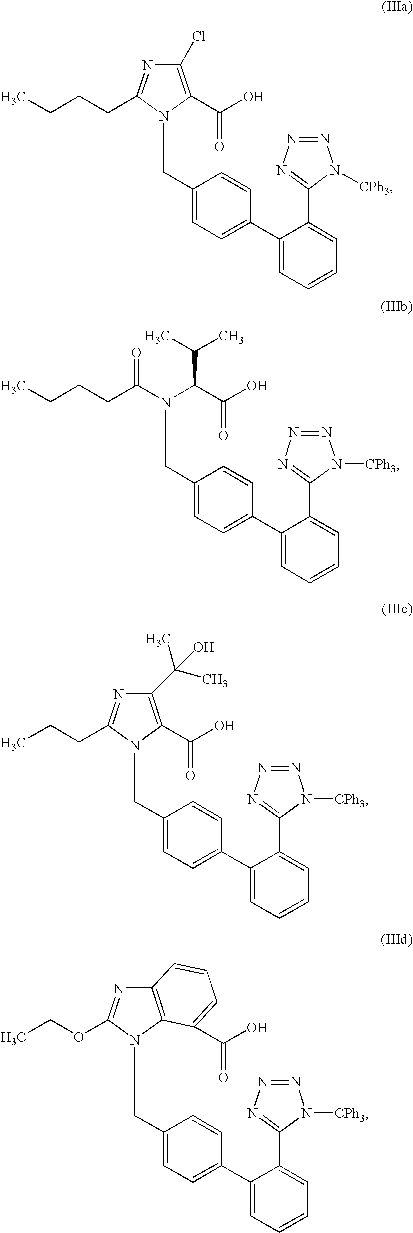

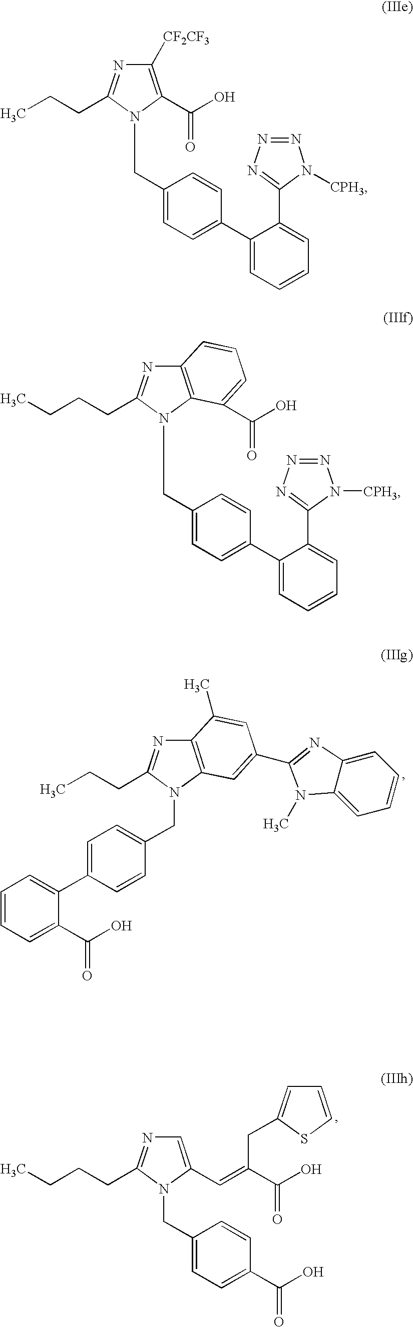

with a compound of formula (IVa) or (IVb), depending on the meaning of Y:

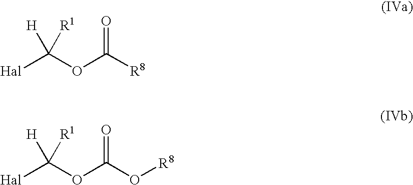

wherein:

R¹ is as above defined;

Hal is a halogen atom like Cl, Br or I;
R⁸ is —(CH₂)ₙ—R⁵ or —(CH₂)ₙ—O—CH₂—R⁵ or is equal to R⁶, wherein n, R⁵ and R⁶ are as above defined;
in the presence of an inorganic or organic base such as Cs₂CO₃, triethylamine or other well known in the literature in an aprotic polar/non polar solvent such as THF, DMF or CH₂Cl₂, at temperatures ranging between 0° C. to 100° C., eventually removing the trityl group when present following methods well known in the art.
Compound (IIIa) is known as trityl EXP 3174 and its preparation is described in Example 1.
Compound (IIIb)-(IIId) are respectively known as trityl valsartan, trityl olmesartan and trityl candesartan and are commercially available.
Compound (IIIe) is known as trityl DuP 532 and can be prepared as described by Michael E. Pierce in J. Org. Chem. 1993, 58, 4642-4645.
Compound (IIIf) can be prepared from a compound known as CV 11194 by reacting with trityl chloride as described for EXP 3174 in Example 1. CV 11194 can be prepared as described by Kubo, K. et al, in J. Med. Chem. 1993, 36, 1772-1784.
Compound (IIIg), (IIIh) are respectively known as telmisartan and eprosartan and are commercially available.
Compound (IIIk) is known as trityl losartan and is commercially available.
Compound (IVa), wherein R¹ and R⁸ are as above defined can be obtained by reacting a compound of formula (Va),

R⁸—COOH (Va)

wherein R⁸ is as above defined, with acetaldehyde in the presence of (COCl)₂ or other condensing agent, and a protic or Lewis acid such as HCl or ZnCl₂ in an aprotic non polar/polar solvent such as CH₂Cl₂, THF or DMF at temperatures ranging between −78° C. to 100° C.
Compound (Va) can be prepared by known compounds by methods well known in the art.
Compound (IVb) wherein R¹ and R⁸ are as above defined can be obtained by reacting commercially available compound (Vb),

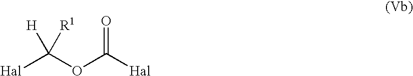

wherein R¹ is as above defined and Hal is an halogen atom like Cl, Br or I, with a compound (VIb)

R⁸—OH (VIb)

wherein R⁸ is as above defined, in the presence of an organic or inorganic base such as pyridine, or triethylamine or other well known in the literature in an aprotic polar/non polar solvent such as THF, DMF or CH₂Cl₂, at temperatures ranging between −78° C. to 40° C.
Compound (VIb) can be obtained following procedures known in the literature.
1.b) R is selected from (IId); R¹═R²═CH₃;
by a process comprising reacting a compound of formula (VII)

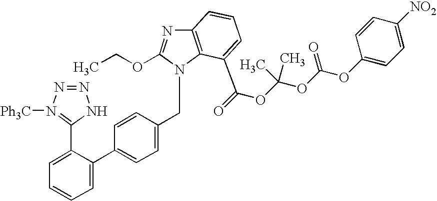

with a compound of formula (VIb) as above defined, in the presence of an organic or inorganic base such as DMAP, Cs₂CO₃, or other well known in the literature, in an aprotic polar/non polar solvent such as THF, DMF or CH₂Cl₂, at temperatures ranging between 0° C. to 100° C., eventually removing the trityl group by known methods.
Compound (VII) can be obtained by reacting compound (IIId) already defined with a compound of formula (VIII):

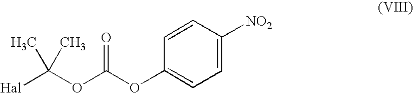

wherein Hal is an halogen such as Cl, Br or I, in the presence of Hg₂O or Ag₂O in an aprotic polar/non polar solvent such as THF, DMF or CH₂Cl₂, at temperatures ranging between 0° C. to 100° C., following methods described by Alexander, J. in PCT. Int. Appl. (1996), WO 9618605
2. R is Selected from (IIk)
The compound of general formula (I) wherein R is selected from (IIk),
and Y is as above defined, can be obtained by a process comprising reacting a compound of formula (IIIk):

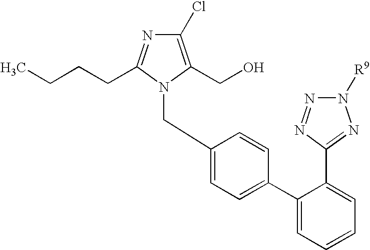

wherein R⁹ is H or the group trityl, with a compound of formula (Va) already defined:

R⁸—COOH (Va)

wherein R⁸ is as above defined, in the presence of a condensing agent like DCC, EDAC, or other well known in the literature, with or without the presence of an inorganic base or organic such as trietylamine, or N-methylmorpholine in an aprotic polar/non polar solvent such as THF, DMF or CH₂Cl₂, at temperatures ranging between 0° C. to 100° C., eventually removing the trityl group when present following methods well known in the art. Alternatively compounds (Va) can be first transformed into acyl chlorides or other activated carboxylic acid esters, such as p-nitrophenyl or pentafluorophenyl esters, following procedures known in the literature and then reacted with compound (IIIk) in the presence of an organic or inorganic base such as TEA, pyridine or DIPEA in an aprotic polar/non polar solvent such as THF, DMF or CH₂Cl₂, at temperatures ranging between 0° C. to 100° C., eventually removing the trityl group when present following methods well known in the art.
The following examples are to further illustrate the invention without limiting it.

### Example 1

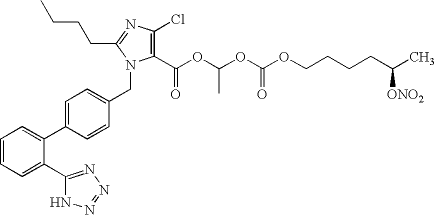

### 1-[({[(R)-5-(nitrooxy)hexyl]oxy}carbonyl)oxy]ethyl 2-butyl-4-chloro-1-{[2′-(1H-tetrazol-5-yl)biphenyl-4-yl]methyl}-1H-imidazole-5-carboxylate

### Step A: (2-butyl-4-chloro-1-{[2′-(1H-tetrazol-5-yl)biphenyl-4-yl]methyl}-1H-imidazole-5-carboxylic acid (E3174)

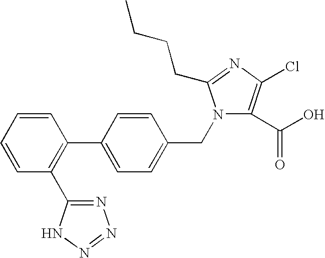

Water (10 L) was added to a 22 L 4-neck round bottom flask. The water was cooled to 0° C. At 0° C., potassium hydroxide (855 g, 15.24 mol) was added followed by losartan potassium (500 g, 1.09 mol), sodium periodate (554 g, 2.59 mol) and ruthenium (III) chloride hydrate (12 g, 0.05 mol) and the reaction mixture was stirred at 0° C. overnight. The reaction mixture was filtered. IPA (90 mL) was added to the filtrate while stirring. The solution was warmed to 25° C. and stirred for 2.5 hrs. After 2.5 hours, phosphoric acid (1200 mL) was added, maintaining the temperature below +30° C. The mixture was stirred for 30 min and the product was filtered, washing with water. The residue was dried in the vacuum oven at 55° C. overnight. The solid was dissolved in methanol (4 L) and isopropyl acetate (12 L), and charcoal (activated carbon) (100 g) was added. The mixture was stirred at rt for 3.5 hrs, filtered and concentrated. The product was redissolved in DCM/MeOH and precipitated with heptane to afford the title compound as a greenish/brown foam which was used in subsequent steps without further purification. Step B: 2-butyl-4-chloro-1-{[2′-(2-trityl-2H-tetrazol-5-yl)biphenyl-4-yl]methyl}-1H-imidazole-5-carboxylic acid

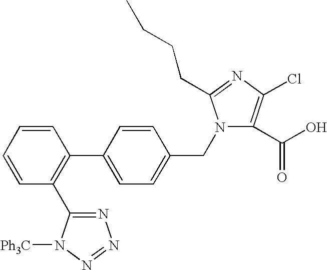

To a solution of E3174 (234.58 g, 0.54 mol) in DCM (4500 mL) was added triethylamine (85 mL, 0.59 mol) followed by a solution of trityl chloride (159 g, 0.56 mol) in DCM (800 mL) and the reaction mixture was stirred at rt overnight. The reaction mixture was washed with water, dried (MgSO4), filtered, and concentrated in vacuo. Chromatography over silica eluting with 20-80% acetone/heptane afforded the title compound as an orange solid.

### Preparation of (5R)-5-(nitrooxy)hexyl-1-chloroethyl carbonate

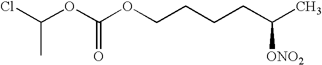

Step A′: (5R)-hexane-1,5-diol
To a solution of (5R)-tert-butyl 5-acetoxyhexanoate (obtained as described in Oscar Pamies and Jan-E. Backvall, J. Org. Chem. 2002, 67, 1261-1265) (5.13 g, 22.3 mmol) in THF (45 mL), cooled to 0° C., LiAlH₄ was added (1M in THF, 45 mL). The reaction was stirred at room temperature for 12 hrs and then quenched with HCl (3N, 2-3 mL). The mixture was diluted with Et₂O (200 mL) and the white precipitate was triturated, filtered off and washed twice with Et₂O. The filtrate was dried with Na₂SO₄ and concentrated affording the title compound.

### Step B′: (5R)-5-hydroxyhexyl 4-nitrobenzoate

(5R)-hexane-1,5-diol was dissolved in CH₂Cl₂ (30 mL) and triethylamine (2.30 mL, 29.9 mmol) and N,N-dimethylaminopyridine (156 mg, 1.27 mmol) were added. The solution was cooled to 0° C. and p-nitrobenzoylchloride (4.74 g, 25.5 mmol) was slowly added. The reaction was stirred at room temperature for 4 hrs, then quenched by addition of NaH₂PO₄ (5%, 30 mL). The organic phase was washed with brine, dried over Na₂SO₄ and concentrated. The crude material was purified by flash chromatography (Biotage SP1, eluting with 10-80% EtOAc/Hexane) affording the title compound

### Step C′: (5R)-5-(nitrooxy)hexyl 4-nitrobenzoate

HNO₃ (fuming, 5 mL) was dissolved in Ac₂O (30 mL) at 0° C. To the solution (5R)-5-hydroxyhexyl 4-nitrobenzoate (4.83 g, 18.1 mmol) was added and the mixture was stirred at 0° C. for 1 h. It was then poured in a mixture of iced NaHCO₃ (100 mL). The aqueous phase was extracted with CH₂Cl₂. The combined organic phases were washed with brine, dried over Na₂SO₄ and concentrated affording the title compound.

### Step D′: (5R)-5-(nitrooxy)hexyl 1-chloroethyl carbonate

(5R)-5-(nitrooxy)hexyl 4-nitrobenzoate (4.68 g, 15.0 mmol) was dissolved in MeOH (60 mL) and NaOH (10%, 60 mL). The reaction was stirred for 1 h at room temperature, the MeOH removed and the aqueous phase extracted with CH₂Cl₂. The combined organic phases were washed with brine, dried over Na₂SO₄ and reduced to a small volume. To the solution pyridine (1.7 mL, 21.0 mmol) was added and, after cooling to −78° C., a CH₂Cl₂ (10 mL) solution of 1-chloroethylchloroformate (2.50 mL, 23.0 mmol). The reaction was warmed to room temperature and stirred for 6 hrs. It was concentrated and purified by flash chromatography (Biotage SP1, eluting with 2-20% EtOAc/Hexane) affording the title compound.

¹H NMR (300 MHz, CDCl₃): δ. 6.45 (1H, m); 5.09 (1H, m); 4.24 (2H, m); 1.85 (3H, dd); 1.75-1.48 (6H, m); 1.38 (3H, d)

### Step C: 1-[({[(R)-5-(nitrooxy)hexyl]oxy}carbonyl)oxy]ethyl 2-butyl-4-chloro-1-{[2′-(1-trityl-1H-tetrazol-5-yl)biphenyl-4-yl]methyl}-1H-imidazole-5-carboxylate

(5R)-5-(nitrooxy)hexyl 1-chloroethyl carbonate (prepared as described in Step D′, 2.09 g, 8.60 mmol), 2-butyl-4-chloro-1-{[2′-(1-trityl-1H-tetrazol-5-yl)biphenyl-4-yl]methyl}-1H-imidazole-5-carboxylic acid (5.90 g, 8.70 mmol) and Cs₂CO₃ (3.90 g, 12.0 mmol) were dissolved in DMF (40 mL) and the reaction was stirred at room temperature for 48 hours.

Then it was partitioned between NaH₂PO₄ (5%, 200 mL) and Et₂O (100 mL). The aqueous phase was extracted with Et₂O and the combined organic phases were washed with brine, dried over Na₂SO₄ and concentrated. The crude material was purified by flash chromatography (Biotage SP1, eluting with 7-60% EtOAc/Hexane) affording the title compound.

### Step D: 1-[({[(R)-5-(nitrooxy)hexyl]oxy}carbonyl)oxy]ethyl 2-butyl-4-chloro-1-{[2′-(1H-tetrazol-5-yl)biphenyl-4-yl]methyl}-1H-imidazole-5-carboxylate

1-[({[(R)-5-(nitrooxy)hexyl]oxy}carbonyl)oxy]ethyl 2-butyl-4-chloro-1-{[2′-(1-trityl-1H-tetrazol-5-yl)biphenyl-4-yl]methyl}-1H-imidazole-5-carboxylate was dissolved in MeOH and treated under microwave irradiation at 90° C. for 20 minutes. The mixture was concentrated and purified by flash chromatography (Biotage SP1, eluting with 1-10% MeOH/CH₂Cl₂) and then by HPLC (Xterra Prep RP18 Column, 5 μm, 19×150 mm, eluting with 70-100% MeCN/water+0.1% HCOOH) affording the title compound as a white solid. ¹H NMR (300 MHz, DMSO): δ. 7.6-7.5 (4H, m); 7.07-6.96 (4H, dd); 6.7 (1H, dd); 5.53 (2H, dd); 5.10 (1H, m); 4.09 (2H, t); 2.62 (2H, t), 1.4-1.7 (9H, m); 1.4-1.2 (8H, m); 0.81 (3H, t).

### Example 2

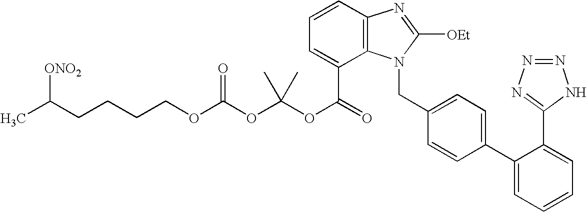

### 1-[({[5-[(nitrooxy)hexyl]oxy}carbonyl)oxy]-1-methylethyl 2-ethoxy-1-{[2′-(1H-tetrazol-5-yl)biphenyl-4-yl]methyl}-1H-benzimidazole-7-carboxylate

### Step A: 1-methyl-1-{[(4-nitrophenoxy)carbonyl]oxy}ethyl 2-ethoxy-1-{[2′-(1-trityl-1H-tetrazol-5-yl)biphenyl-4-yl]methyl}-1H-benzimidazole-7-carboxylate

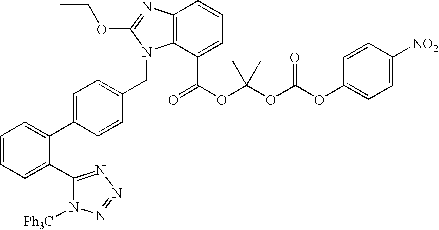

An orange suspension of mercuric oxide (1.17 g, 5.39 mmol) and 2-ethoxy-1-{[2′-(1-trityl-1H-tetrazol-5-yl)biphenyl-4-yl]methyl}-1H-benzimidazole-7-carboxylic acid (7.36 g, 10.8 mmol) in dry tetrahydrofuran (95 mL) was stirred at room temperature for 24 hours. Then 2-chloroisopropyl p-nitrophenyl carbonate (prepared as described in U.S. Pat. No. 5,684,018) (1.40 g, 5.39 mmol) was added, and the reaction was stirred at room temperature for about 7 days and monitored by TLC (hexane/ethyl acetate 6/4). The mixture was diluted with dichloromethane, washed with water, and the organic layer was dried over sodium sulfate and concentrated under reduced pressure. The residue was purified by flash chromatography (Biotage SP1, eluting with 7-60% Ethyl acetate/Hexane), affording the title product. ¹H-NMR ((300 MHz, CDCl₃): δ. 8.23 (2H, d), 7.89-7.83 (1H, m), 7.80 (1H, d), 7.64 (1H, d), 7.51-7.40 (2H, m), 7.38-7.15 (13H, m), 7.02-6.88 (8H, m), 6.74 (1H, d), 5.63 (2H, s), 4.65 (2H, q), 1.83 (6H, s), 1.44 (3H, t).

### Preparation of 5-(nitrooxy)hexanol

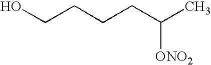

### Step A′: 5-(nitrooxy)hexyl 4-nitrobenzoate

1,5-hexandiol (1.00 mL, 8.30 mmol), triethylamine (1.63 mL, 12.8 mmol) and N,N-dimethylaminopyridine (53.0 mg, 0.43 mL) were dissolved in CH₂Cl₂ (15 mL) and to the solution, cooled to 0° C., p-nitrobenzoyl chloride (1.92 g, 10.3 mmol) was added. The reaction was slowly warmed and stirred overnight, then it was washed with NaH₂PO₄ and brine, dried over Na₂SO₄, and concentrated to a small volume (10 mL). Tetraethylammonium nitrate (3.40 g, 17.7 mmol) and 2,6-di-tert-butyl-4-methylpyridine (2.72 g, 13.2 mmol) were added and the solution was cooled to −78° C. A solution of triflic anhydride (1.37 mL, 8.30 mmol) in CH₂Cl₂ (15 mL) was slowly added. The reaction was stirred at −78° C. for 1 hour and then slowly warmed to room temperature and stirred for 2 hours. The mixture was washed with NaH₂PO₄ and brine, dried over Na₂SO₄ and concentrated. The crude material was purified by flash chromatography (Biotage SP1, eluting with 5-40% EtOAc/Hexane) affording the title compound.

### Step B′: 5-(nitrooxy)hexanol

The residue obtained in Step A′ was dissolved in MeOH (10.0 mL). NaOH (10%, 5 mL) was added and the mixture was stirred at room temperature for 3 hours. MeOH was removed and the aqueous phase was extracted with CH₂Cl₂ (3×10 mL). The organic phase was washed with brine, dried over Na₂SO₄ and concentrated affording the title compound.

### Step B: 1-[({[5-(nitrooxy)hexyl]oxy}carbonyl)oxy]-1-methylethyl 2-ethoxy-1-{[2′-(1-trityl-1H-tetrazol-5-yl)biphenyl-4-yl]methyl}-1H-benzimidazole-7-carboxylate

5-(nitrooxy)hexanol (320 mg, 1.90 mmol) was dissolved in CH₂Cl₂ (15 mL) and to the solution N,N-dimethylaminopyridine (170 mg, 1.40 mmol) and 1-methyl-1-{[(4-nitrophenoxy)carbonyl]oxy}ethyl 2-ethoxy-1-{[2′-(1-trityl-1H-tetrazol-5-yl)biphenyl-4-yl]methyl}-1H-benzimidazole-7-carboxylate (1.25 g, 1.40 mmol) were added. The reaction was stirred at room temperature for 12 hours, then washed with NaH₂PO₄ and brine, dried over Na₂SO₄ and concentrated. The crude material was purified by flash chromatography (Biotage SP1, eluting with 7-60% EtOAc/Hexane) affording the title compound.

### Step C: 1-[({[5-(nitrooxy)hexyl]oxy}carbonyl)oxy]-1-methylethyl 2-ethoxy-1-{[2′-(1H-tetrazol-5-yl)biphenyl-4-yl]methyl}-1H-benzimidazole-7-carboxylate

1-[({[5-(nitrooxy)hexyl]oxy}carbonyl)oxy]-1-methylethyl 2-ethoxy-1-{[2′-(1-trityl-1H-tetrazol-5-yl)biphenyl-4-yl]methyl}-1H-benzimidazole-7-carboxylate was dissolved in MeOH (20 mL) and irradiated with microwaves (90° C., 20 minutes). The mixture was concentrated and purified first by flash chromatography (Biotage SP1, eluting with 1-10% MeOH/CH₂Cl₂), then by HPLC (Xterra Prep RP18 Column, 5 μm, 19×150 mm, eluting with 70-100% MeCN/water+0.1% HCOOH) affording the title compound as a white solid. ¹H NMR (300 MHz, DMSO): δ. 7.75-7.47 (6H, m); 7.25-7.18 (1H, t); 7.0 (2H, d); 6.88 (2H, d); 5.53 (2H, s); 5.11-4.99 (1H, m); 4.61 (2H, q); 4.06 (2H, t); 1.75 (6H, s); 1.65-1.5 (4H, m); 1.45-1.27 (5H, m); 1.21 (3H, d).

### Intermediate 1

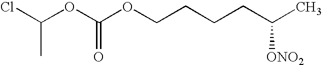

### (5S)-5-(nitrooxy)hexyl 1-chloroethyl carbonate

The title compound was prepared by following Step A′ in example 1, except that the reagent (5R)-tert-butyl-5-acetoxyhexanoate was replaced by (5S)-tert-butyl-5-hydroxyhexanoate (also obtained as described in Oscar Pamies and Jan-E. Backvall, J. Org. Chem. 2002, 67, 1261-1265).

### Intermediate 2

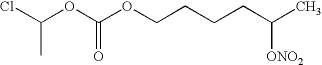

### 5-(nitrooxy)hexyl 1-chloroethyl carbonate

The title compound was prepared by following Step B′ in example 1, except that the reagent (5R)-hexane-1,5-diol was replaced by commercially available hexane-1,5-diol.

### Intermediate 3

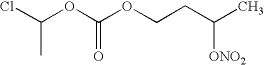

### 3-(nitrooxy)butyl 1-chloroethyl carbonate

The title compound was prepared by following Step B′ in example 1, except that the reagent (5R)-hexane-1,5-diol was replaced by commercially available butane-1,3-diol.

¹H NMR (300 MHz, CDCl₃): δ. 6.43 (1H, m); 5.24 (1H, m); 4.33 (2H, m); 2.06 (2H, m); 1.86 (3H, d); 1.44 (3H, d);

### Intermediate 4

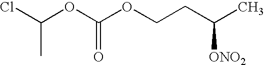

### (3R)-3-(nitrooxy)butyl 1-chloroethyl carbonate

The title compound was prepared by following Step B′ in example 1, except that the reagent (5R)-hexane-1,5-diol was replaced by commercially available (3R)-butane-1,3-diol.

### Intermediate 5

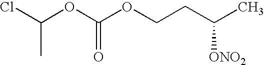

### (3S)-3-(nitrooxy)butyl 1-chloroethyl carbonate

The title compound was prepared by following Step B′ in example 1, except that the reagent (5R)-hexane-1,5-diol was replaced by commercially available (3S)-butane-1,3-diol.

### Intermediate 6

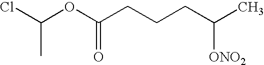

### 1-chloroethyl 5-(nitrooxy)hexanoate

### Step A′: tert-butyl 5-(nitrooxy)hexanoate

tert-butyl-5-hydroxyhexanoate (obtained as described in Oscar Pamies and Jan-E. Backvall, J. Org. Chem. 2002, 67, 1261-1265) (3.81 g, 20.2 mmol), 2,6-di-tert-butyl-4-methyl pyridine (6.64 g, 32.2 mmol), tetraethylammonium nitrate (7.76 g, 40.4 mmol) were dissolved in CH₂Cl₂ (75 mL). To the solution, cooled to −78° C., a solution of triflic anhydride (3.33 ml, 20.2 mmol) in CH₂Cl₂ (75 mL) was slowly added. The reaction was stirred at −78° C. for 30 minutes and then slowly warmed to room temperature and stirred for three hours. The mixture was then washed with NaH₂PO₄ and brine, dried over Na₂SO₄ and concentrated. The crude material was purified by flash chromatography (Biotage SP1, eluting with 2-20% EtOAc/Hexane) affording the title compound.

### Step B′: 1-chloroethyl 5-(nitrooxy)hexanoate

HCl_{gas} was bubbled into a solution of tert-butyl-5-(nitrooxy)hexanoate (590 mg, 2.53 mmol) in CH₂Cl₂ (20 mL). After the disappearance of the starting material, (COCl)₂ (110 μL, 1.25 mmol) and DMF (few drops) were added. Once ceased the gas evolution ZnCl₂ (86.0 mg, 0.63 mmol) was added and the solution was cooled to 0° C. Acetaldehyde (1.54 mL, 27.2 mmol) was added and the reaction was stirred at room temperature overnight, then concentrated and purified by flash chromatography (Biotage SP1, eluting with 2-20% EtOAc/Hexane) affording the title compound.

¹H NMR (300 MHz, CDCl₃): δ. 6.56 (1H, q); 5.10 (1H, m); 6.7 (1H, dd); 2.38 (2H, m); 1.80 (3H, d); 1.72 (4H, m); 2.62 (2H, t), 1.38 (3H, d)

### Intermediate 7

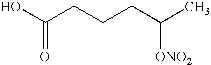

### 5-(nitrooxy)hexanoic acid

tert-butyl-5-(nitrooxy)hexanoate (590 mg, 2.53 mmol), prepared following Step A′ in Intermediate 6, was dissolved in CH₂Cl₂ (20 mL). HCl_{gas} was bubbled into the solution until the disappearance of the starting material. The solution was reduced to a small volume and diluted in CH₂Cl₂ few times to remove residual acidity, then used without further purification.

¹H NMR (300 MHz, CDCl₃): δ. 9.01 (1H, bs); 5.09 (1H, m); 2.43 (2H, t); 1.70 (4H, m); 1.37 (3H, d)

### Example 3

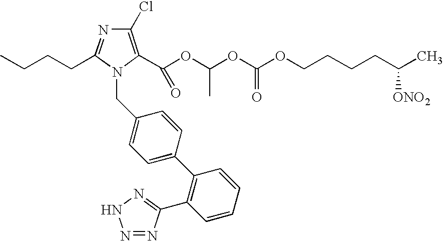

### 1-[({[(5S)-5-(nitrooxy)hexyl]oxy}carbonyl)oxy]ethyl 2-butyl-4-chloro-1-{[2′-(1H-tetrazol-5-yl)biphenyl-4-yl]methyl}-1H-imidazole-5-carboxylate

Title compound was synthesized by following Step C in example 1, except that the reagent (5R)-5-(nitrooxy)hexyl 1-chloroethyl carbonate was replaced by (5S)-5-(nitrooxy)hexyl 1-chloroethyl carbonate (Intermediate 1).

### Example 4

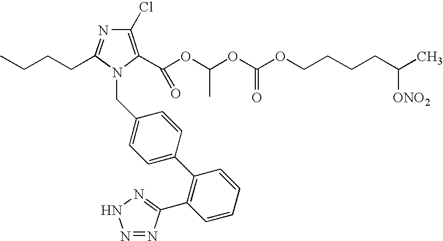

### 1-[({[5-(nitrooxy)hexyl]oxy}carbonyl)oxy]ethyl 2-butyl-4-chloro-1-{[2′-(1H-tetrazol-5-yl)biphenyl-4-yl]methyl}-1H-imidazole-5-carboxylate

Title compound was synthesized by following Step C in example 1, except that the reagent (5R)-5-(nitrooxy)hexyl-1-chloroethyl carbonate was replaced by 5-(nitrooxy)hexyl 1-chloroethyl carbonate (Intermediate 2).

### Example 5

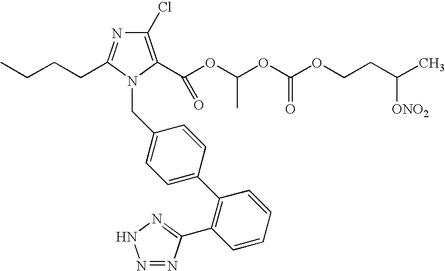

### 1-[({[3-(nitrooxy)butyl]oxy}carbonyl)oxy]ethyl 2-butyl-4-chloro-1-{[2′-(1H-tetrazol-5-yl)biphenyl-4-yl]methyl}-1H-imidazole-5-carboxylate

Title compound was synthesized by following Step C in example 1, except that the reagent (5R)-5-(nitrooxy)hexyl 1-chloroethyl carbonate was replaced by 3-(nitrooxy)butyl 1-chloroethyl carbonate (Intermediate 3).

¹H NMR (300 MHz, DMSO): δ. 7.7-7.47 (4H, m); 7.08 (2H, d); 6.98 (2H, d); 6.77 (1H, q); 5.54 (2H, q); 5.27-5.11 (1H, m); 4.22-4.17 (2H, m); 2.62 (2H, t); 2.05-1.95 (2H, m); 1.6-1.45 (5H, m); 1.37-1.19 (5H, m); 0.8 (3H, t).

### Example 6

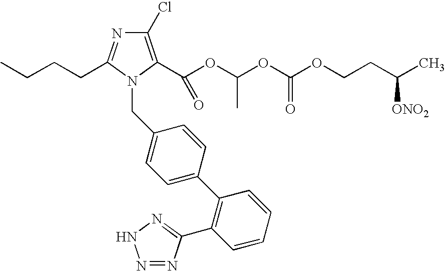

### 1-[({[(3R)-3-(nitrooxy)butyl]oxy}carbonyl)oxy]ethyl 2-butyl-4-chloro-1-{[2′-(1H-tetrazol-5-yl)biphenyl-4-yl]methyl}-1H-imidazole-5-carboxylate

Title compound was synthesized by following Step C in example 1, except that the reagent (5R)-5-(nitrooxy)hexyl 1-chloroethyl carbonate was replaced by (3R)-3-(nitrooxy)butyl 1-chloroethyl carbonate (Intermediate 4).

### Example 7

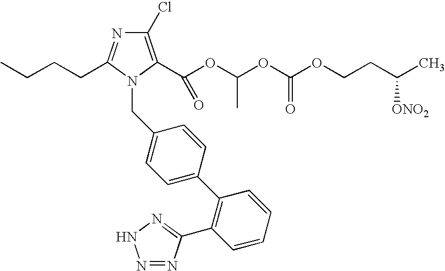

### 1-[({[(3S)-3-(nitrooxy)butyl]oxy}carbonyl)oxy]ethyl 2-butyl-4-chloro-1-{[2′-(1H-tetrazol-5-yl)biphenyl-4-yl]methyl}-1H-imidazole-5-carboxylate

Title compound was synthesized by following Step C in Example 1, except that the reagent (5R)-5-(nitrooxy)hexyl 1-chloroethyl carbonate was replaced by (3S)-3-nitrooxybutyl 1-chloroethyl carbonate (Intermediate 5).

### Example 8

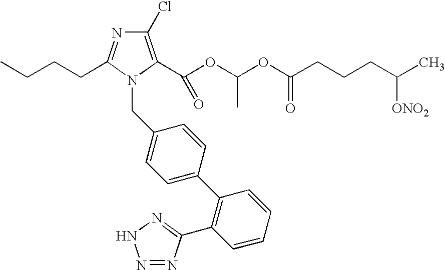

### 1-({[5-(nitrooxy)hexyl]carbonyl}oxy)ethyl 2-butyl-4-chloro-1-{[2′-(1H-tetrazol-5-yl)biphenyl-4-yl]methyl}-1H-imidazole-5-carboxylate

Title compound was synthesized by following Step C in Example 1, except that the reagent (5R)-5-(nitrooxy)hexyl 1-chloroethyl carbonate was replaced by 1-chloroethyl 5-(nitrooxy)hexanoate (Intermediate 6). (DMSO): 7.71-7.49 (4H, m); 7.1-7.05 (2H, d); 7.0-6.92 (2H, d); 6.9-6.82 (1H, m); 5.61-5.49 (2H, m); 5.15-5.05 (1H, m); 2.65-2.59 (2H, m); 2.4-2.3 (2H, m); 1.65-1.4 (9H, m); 1.32-1.19 (5H, m); 0.72 (3H, t).

### Example 9

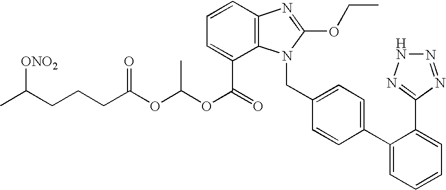

### 1-({[5-(nitrooxy)hexyl]carbonyl}oxy)ethyl 2-ethoxy-1-{[2′-(1H-tetrazol-5-yl]-biphenyl-4-yl}-methyl}-1H-benzimidazole-7-carboxylate

### Step A: 1-({[5-(nitrooxy)hexyl]carbonyl}oxy)ethyl 2-ethoxy-1-{[2′-(1-trityl-1H-tetrazol-5-yl]-biphenyl-4-yl]-methyl}-1H-benzimidazole-7-carboxylate

Title compound was prepared by following Step C in example except that the reagent 2-butyl-4-chloro-1-{[2′-(2-trityl-2H-tetrazol-5-yl)biphenyl-4-yl]methyl}-1H-imidazole-5-carboxylic acid was replaced by 2-ethoxy-1-[{2′-[1-(trityl)-1H-tetrazol-5-yl]-biphenyl-4-yl}-methyl]-1H-benzimidazole-7-carboxylic acid, and the reagent (5R)-5-(nitrooxy)hexyl 1-chloroethyl carbonate was replaced by 1-chloroethyl 5-(nitrooxy)hexanoate (Intermediate 6). ¹H NMR (300 MHz, DMSO): δ. 7.79-7.7 (1H, m); 7.69-7.5 (3H, m); 7.5-7.41 (1H, m); 7.2 (1H, t); 7.05-6.85 (5H, m); 5.6-5.4 (2H, m); 5.15-5.05 (1H, m); 4.6 (2H, q); 2.3-2.4 (2H, m); 1.7-1.5 (4H, m); 1.42-1.32 (6H, m); 1.25 (3H, d).

### Example 10

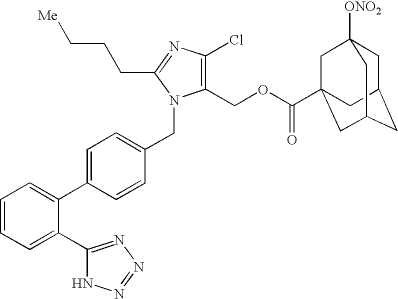

### (2-butyl-4-chloro-1-{[2′-(1H-tetrazol-5-yl)biphenyl-4-yl]methyl}-1H-imidazol-5-yl)methyl 3-(nitrooxy)adamantane-1-carboxylate

### Step A: 3-(nitrooxy)adamantane-1-carboxylic acid

To an acetic anhydride (50 mL) solution of nitric acid (3 mL, 47.0 mmol) at 0° C. was added 3-hydroxyadamantane-1-carboxylic acid (5.56 g, 28.3 mmol). After 30 minutes, the reaction mixture was quenched into a saturated ice-bath of sodium bicarbonate. Hydrochloric acid was added to bring the pH to around 4-5. The reaction mixture was extracted with ethyl acetate, and the combined organic extracts were dried (magnesium sulfate) and concentrated in vacuo to afford a white solid. Chromatography over silica gel, eluting with 98/2→88/12 dichloromethane/methanol, afforded the title compound as a white solid. ¹H NMR (500 MHz, CDCl₃) δ. 2.41-2.48 (m, 2H), 2.32-2.36 (m, 2H), 2.12-2.20 (m, 4H), 1.88-1.98 (m, 4H), 1.68-1.76 (m, 2H).

### Step B: (2-butyl-4-chloro-1-{[2′-(1H-tetrazol-5-yl)biphenyl-4-yl]methyl}-1H-imidazol-5-yl)methyl 3-(nitrooxy)adamantane-1-carboxylate

To a dichloromethane (30 mL) suspension of losartan potassium (0.515 g, 1.12 mmol) and 3-(nitrooxy)adamantane-1-carboxylic acid (0.195 g, 0.810 mmol) was added N-methylmorpholine (0.267 mL, 2.43 mmol), followed by 1-ethyl-3-(3′-dimethylaminopropyl)carbodiimide hydrochloride (0.194 g, 1.01 mmol). After 2 days, the reaction mixture was concentrated in vacuo, and purification of the residue by reversed-phase mass-directed high-performance liquid chromatography afforded the title compound. ¹H NMR (500 MHz, CDCl₃) δ 7.91 (d, J=7.5 Hz, 1H), 7.61 (dt, J=1.4, 7.6 Hz, 1H), 7.55 (dt, J=1.1, 7.6 Hz, 1H), 7.43 (d, J=7.6 Hz, 1H), 7.14 (d, J=8.2 Hz, 2H), 6.82 (d, J=8.0 Hz, 2H), 5.14 (s, 2H), 4.86 (s, 2H), 2.40 (t, J=7.8 Hz, 2H), 2.29-2.37 (m, 2H), 1.98-2.09 (m, 4H), 1.65-1.78 (m, 4H), 1.56-1.66 (m, 4H), 1.24-1.36 (m, 4H), 0.86 (t, J=7.3 Hz, 3H); LC-MS: m/z 646.1 (M+H).

1. A compound having the general formula 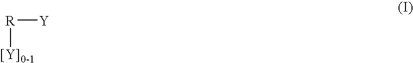
wherein R is selected from the group consisting of (IIa)-(IIk): 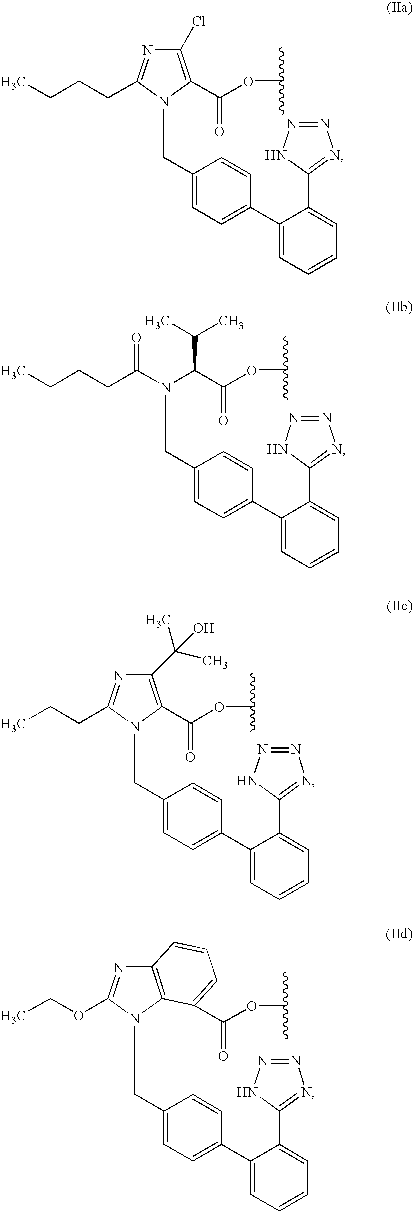 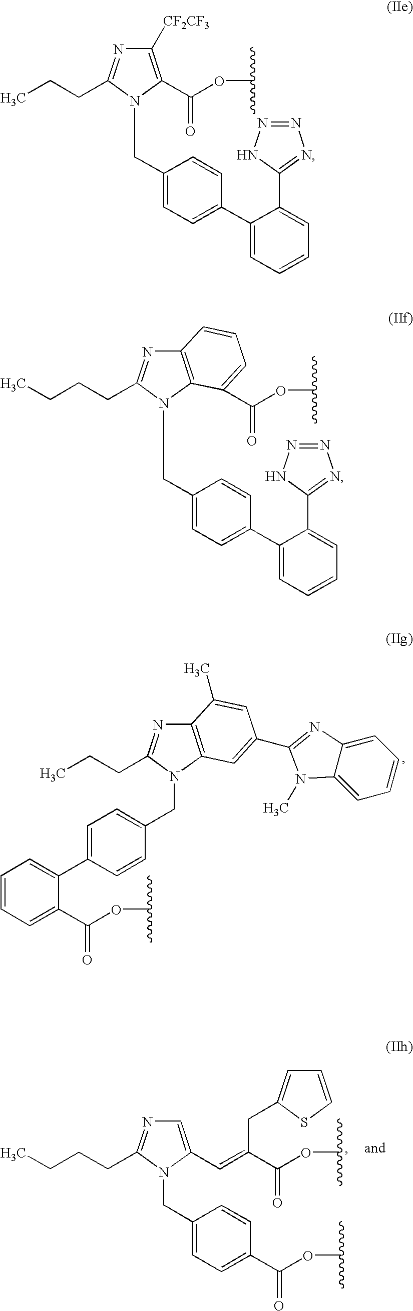 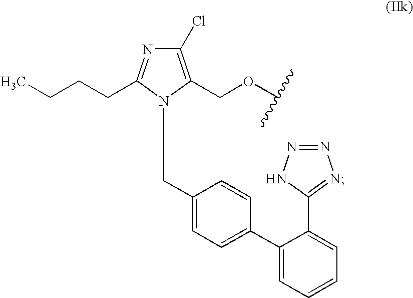
when R is selected from (IIk), Y is selected from the group consisting of:
1) —C(O)(CH₂)ₙR⁵
2) —C(O)(CH₂)ₙ—O—CH₂—R⁵,
3) —C(O)—R⁶ wherein R⁶ is the following group: 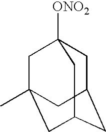
when R is selected from (IIa)-(IIh), Y is selected from the group consisting of
4) —C(R¹R²)OC(O)—(CH₂)ₙR⁵,
5) —C(R¹R²)OC(O)O—(CH₂)ₙR⁵,
6) C(R¹R²)OC(O)(CH₂)ₙ—O—CH₂—R⁵,
7) C(R¹R²)OC(O)O(CH₂)ₙ—O—CH₂—R⁵
8) —C(R¹R²)OC(O)—R⁶ wherein R is as above defined;
R¹ and R², are independently selected from the group consisting of hydrogen and C₁₋₄ alkyl;
R⁵ is —CH(ONO₂)R⁷;
R⁷ is CH₃ or C₁₋₄ alkyl;
n is an integer from 1 to 4;
or a pharmaceutically acceptable salt thereof.

2. A compound of claim 1, wherein R¹ is CH₃ and R² is H or CH₃.

3. A compound of claim 1, wherein R is selected from the group consisting of (IIb), (IIc) and (IIg):

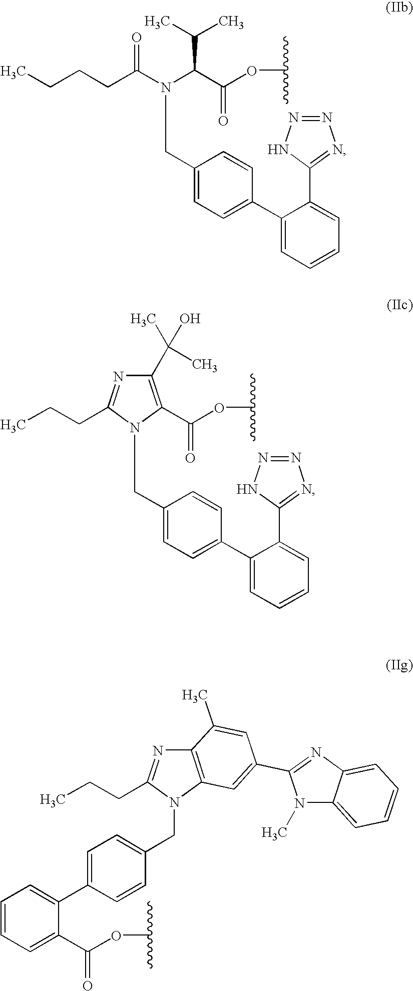

4. A compound of claim 1, wherein R is selected from the group consisting of (IIa), (IId), and (IIk):

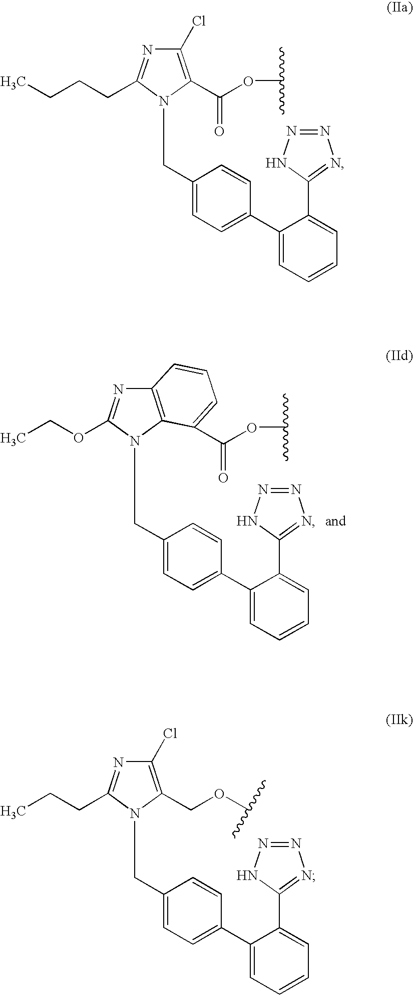

5. A compound of claim 1, wherein R⁵ is selected from the group consisting of

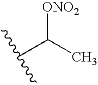

6. A compound of claim 1, wherein R⁵ is selected from the group consisting of

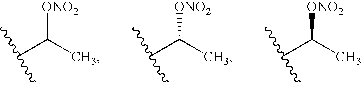

7. A compound of claim 1, having the formula 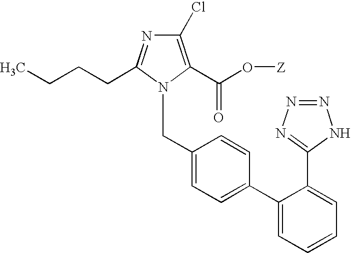
wherein Z is selected from the group consisting of 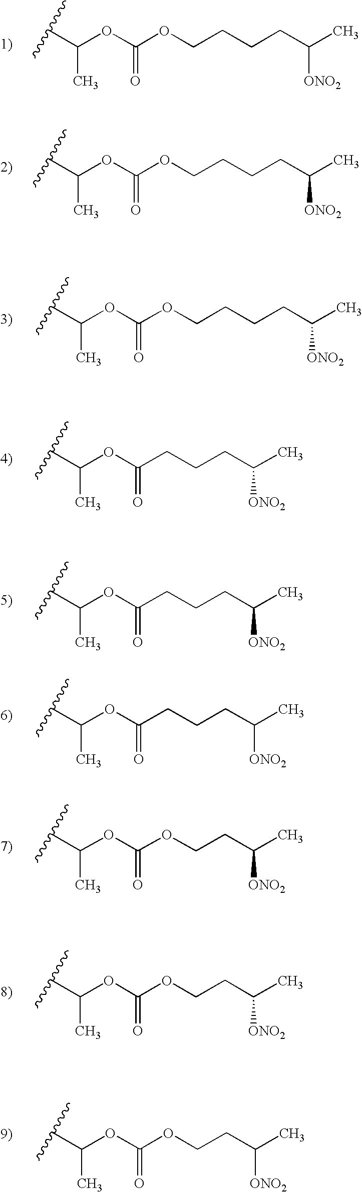
or a pharmaceutically acceptable salt thereof.

8. A compound of claim 1, having the formula 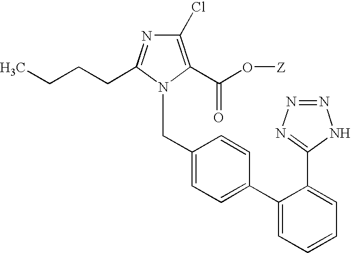
wherein Z is selected from the group consisting of 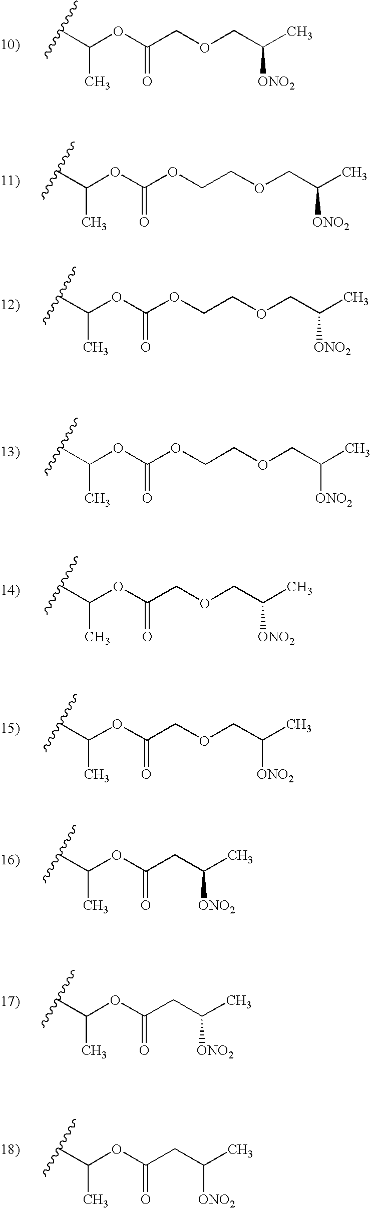
or a pharmaceutically acceptable salt thereof.

9. A compound of claim 1, having the formula 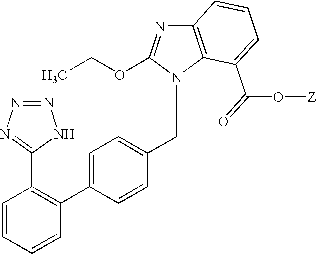
wherein Z is selected from the group consisting of 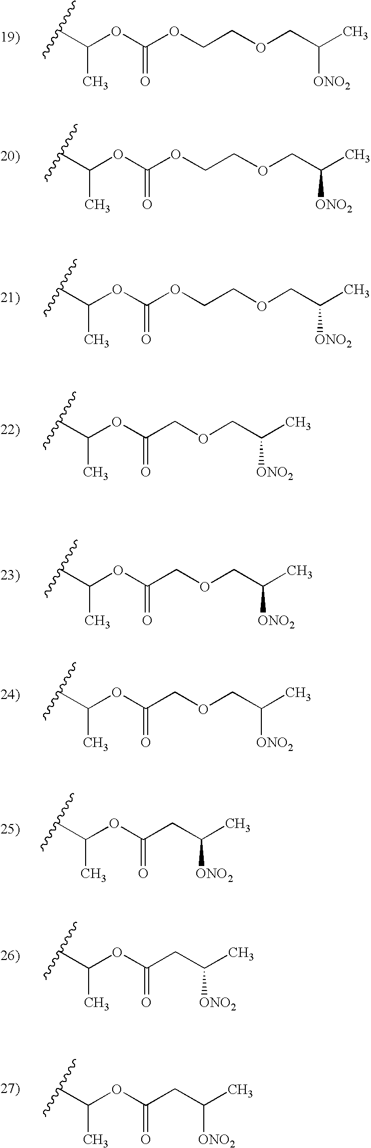
or a pharmaceutically acceptable salt thereof.

10. A compound of claim 1, having the formula 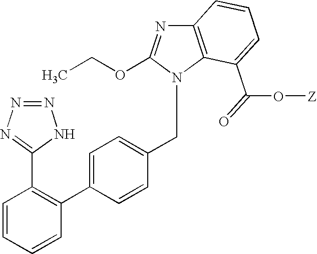
wherein Z is selected from the group consisting of 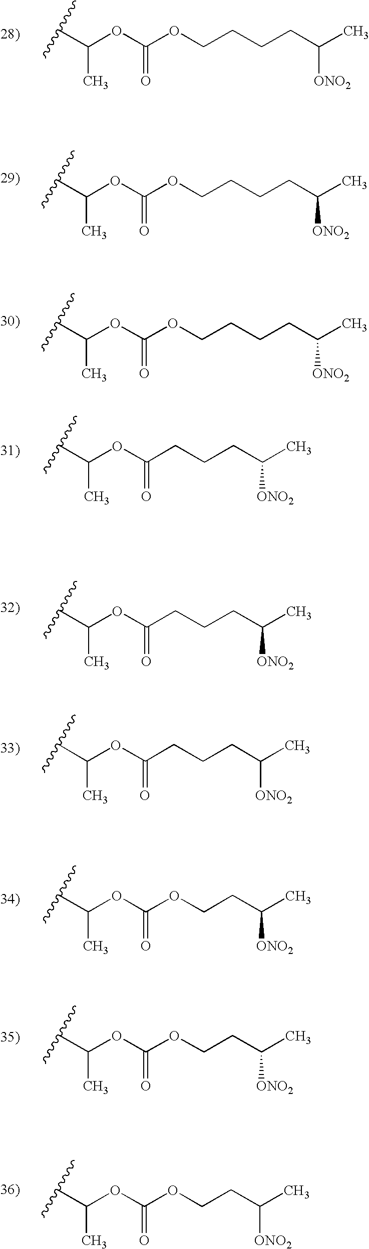
or a pharmaceutically acceptable salt thereof.

11. A compound of claim 1, having the formula 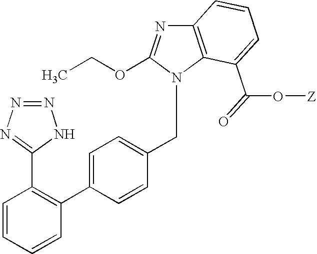
wherein Z is selected from the group consisting of 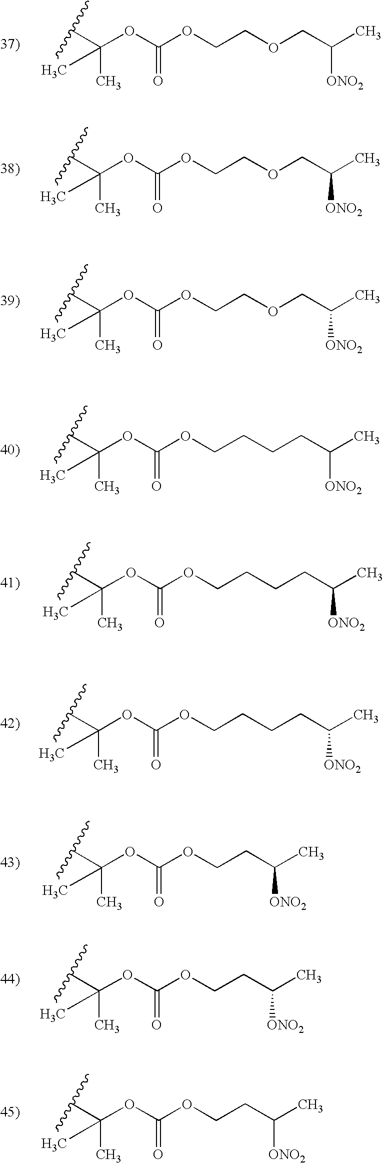
or a pharmaceutically acceptable salt thereof.

12. A compound of claim 1, having the formula 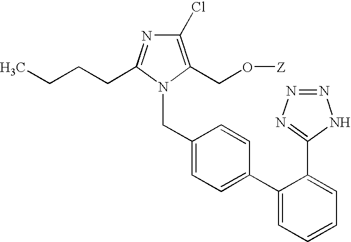
wherein Z is selected from the group consisting of 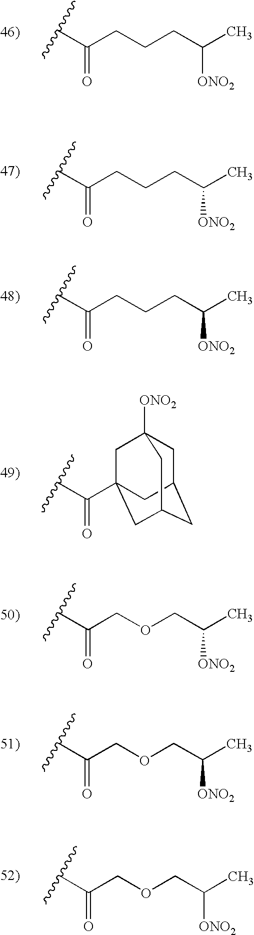
or a pharmaceutically acceptable salt thereof.

13. A pharmaceutical composition comprising a compound of claim 1 and a pharmaceutically acceptable carrier.

14. A pharmaceutical composition comprising a compound of claim 1, a diuretic, and a pharmaceutically acceptable carrier.

15. Use of a compound of claim 1 for treating hypertension.
